(19) United States
(12) Patent Application Publication
(10) Pub. No.: US 2023/0253113 A1
(43) Pub. Date: Aug. 10, 2023

(54) SYSTEMS AND METHODS FOR CREATING BIOMOLECULE EMBEDDINGS

(71) Applicant: Seer, Inc., Redwood City, CA (US)

(72) Inventors: Harendra GUTURU, Oakland, CA (US); Mahdi ZAMANIGHOMI, Belmont, MA (US)

(21) Appl. No.: 18/164,542

(22) Filed: Feb. 3, 2023

Related U.S. Application Data
(60) Provisional application No. 63/306,958, filed on Feb. 4, 2022.
(60) Provisional application No. 63/310,453, filed on Feb. 15, 2022.

(51) Int. Cl. G16H 50/20; G16B 20/00; G16B 40/10; G16B 40/20

(52) U.S. Cl. CPC G16H 50/20; G16B 20/00; G16B 40/10; G16B 40/20

(57) ABSTRACT

In some aspects, the present disclosure describes a method for determining a biological state associated with a polyamino acid descriptor. In some cases, the method comprises receiving the polyamino acid descriptor comprising at least one dimension representing a polyamino acid association with a given assay method. In some cases, the method comprises generating, in a latent space, a latent descriptor based at least in part on the polyamino acid descriptor, and wherein the latent descriptor comprises sufficiently fewer dimensions than the polyamino acid descriptor such that at least a portion of information in the polyamino acid descriptor is lost in the latent descriptor. In some cases, the method comprises determining, based at least in part on the latent descriptor, the biological state associated with the polyamino acid descriptor.

### CROSS-REFERENCE

This application claims the benefit of U.S. Provisional Patent Application No. 63/306,958, filed Feb. 4, 2022, and U.S. Provisional Patent Application No. 63/310,453, filed Feb. 15, 2022, each of which is incorporated herein by reference in its entirety.

### BACKGROUND

Early detection of disease (e.g., cancer) is key to a favorable prognosis, but there has been little progress in the development of useful clinical tests. Biomolecules in plasma should be a valuable biomarker discovery matrix given plasma's contact with almost all tissues in the body. However, plasma proteins can be problematic to characterize due to several factors including a wide range of concentration (e.g., 10-orders of magnitude). Complex biochemical workflows have attempted to circumvent these challenges but may not be practical for discovery studies of sufficient size to ensure validation and replication. Alternatively, biomarker studies have been limited to evaluating or re-evaluating known markers without substantive improvement in clinical performance.

### SUMMARY

Disclosed herein are systems and methods for analyzing biomolecule-surface interactions. Interactions between biomolecules and surfaces may provide insights on the association of certain bimolecular signatures with biological states across samples.

An aspect of the present disclosure provides a method for training a neural network, comprising: providing a neural network comprising: an input layer configured to receive at least a polyamino acid descriptor; a latent layer configured to output at least a latent descriptor, wherein the latent layer is connected to the input layer, and wherein the latent descriptor comprises sufficiently fewer dimensions than the polyamino acid descriptor such that at least a portion of information in the polyamino acid descriptor is filtered in the latent descriptor; an output layer configured to output at least a reconstruction of the polyamino acid descriptor, wherein the output layer is connected to the latent layer; and at least one parameter; providing training data comprising a plurality of polyamino acid descriptors, wherein the plurality of polyamino acid descriptors comprises at least one value for a polyamino acid in association with a given assay method; training the neural network, by (i) inputting at least the plurality of polyamino acid descriptors at the input layer of the neural network, (ii) outputting a plurality of latent descriptors at the latent layer and a plurality of reconstructions at the output layer, and (iii) optimizing at least one loss function based at least in part on the plurality of latent descriptors and the plurality of reconstructions by adjusting the at least one parameter, such that the neural network learns a latent space comprising a denoised embedding for the plurality of polyamino acid descriptors.

In some embodiments, the output layer outputs a plurality of parameters for a probability distribution. In some embodiments, the probability distribution is a zero inflated distribution. In some embodiments, the zero inflated distribution is a zero inflated negative binomial distribution. In some embodiments, the portion of information comprises noise. In some embodiments, the polyamino acid descriptor comprises at least 100 dimensions. In some embodiments, the latent descriptor comprises at most about 50% of the number of dimensions in the polyamino acid descriptor. In some embodiments, the plurality of polyamino acid descriptors in the training data comprises more zero values than non-zero values. In some embodiments, at least about 10% of values in the plurality of polyamino acid descriptors in the training data are zero. In some embodiments, the at least one loss function comprises a reconstruction loss based at least in part on a difference between the plurality of reconstructions and the plurality of polyamino acid descriptors. In some embodiments, the latent layer outputs a plurality of parameters for a posterior distribution. In some embodiments, the posterior distribution is a Laplace distribution. In some embodiments, the at least one loss function comprises a Kullbeck-Leibler divergence loss function based at least in part on a difference between a sum of posterior distributions parameterized by the plurality of parameters and a prior distribution. In some embodiments, the prior distribution is tighter than a normal distribution. In some embodiments, the prior distribution comprises a higher kurtosis than a normal distribution. In some embodiments, the prior distribution is a Laplace distribution. In some embodiments, optimizing the at least one loss function is performed through gradient descent, such that the at least one parameter of the neural network is updated, wherein the plurality of parameters for the posterior distribution is based at least in part on the at least one parameter of the neural network. In some embodiments, the given assay method comprises contacting a plurality of biomolecules with a given surface. In some embodiments, the given surface is a surface of a particle. In some embodiments, the given assay method comprises (i) performing mass spectrometry on cleaved derivatives of the plurality of biomolecules to obtain a plurality of peptide spectral signals and (ii) processing the plurality of peptide spectral signals to obtain a plurality of peptide identifications, wherein the plurality of polyamino acid descriptors comprises the plurality of peptide identifications. The method claim 18, wherein the given assay method comprises (i) performing mass spectrometry on cleaved derivatives of the plurality of biomolecules to obtain a plurality peptide spectral signals (ii) processing the plurality of peptide spectral signals to obtain a plurality of peptide identifications and (iii) processing the plurality of peptide identifications to obtain a plurality of intensities for plurality of protein or protein group identification, wherein the plurality of polyamino acid descriptors comprises the plurality of protein or protein group identifications. In some embodiments, the method further comprises classifying at least a first set of latent descriptors from a second set of latent descriptors, wherein the first set of latent descriptors is associated with a first biological state and the second set of latent descriptors is associated with a second biological state. In some embodiments, the method further comprises obtaining a set of polyamino acid descriptors corresponding to the first set of latent descriptors. In some embodiments, the set of polyamino acid descriptors comprises at least one polyamino acid descriptor. In some embodiments, the at least one polyamino acid descriptor comprises an identification of at least one protein or protein group. In some embodiments, the at least one polyamino acid descriptor comprises an identification of at least one peptide.

In another aspect, the present disclosure provides a computer-implemented method, comprising implementing any one of the methods for training a neural network disclosed herein in a computer.

In another aspect, the present disclosure provides a computer program product comprising a computer-readable medium having computer-executable code encoded therein, the computer-executable code adapted to be executed to implement any one of the methods for training a neural network disclosed herein.

In another aspect, the present disclosure provides a non-transitory computer-readable storage media encoded with a computer program including instructions executable by one or more processors to learn a denoised embedding using any one of the methods disclosed herein.

In another aspect, the present disclosure provides a computer-implemented system comprising: a digital processing device comprising: at least one processor, an operating system configured to perform executable instructions, a memory, and a computer program including instructions executable by the digital processing device to learn a denoised embedding using any one of the methods of disclosed herein.

An aspect of the present disclosure provides a method for determining a biological state associated with a polyamino acid descriptor, comprising: receiving the polyamino acid descriptor comprising at least one dimension representing a polyamino acid association with a given assay method; generating, in a latent space, a latent descriptor based at least in part on the polyamino acid descriptor, and wherein the latent descriptor comprises sufficiently fewer dimensions than the polyamino acid descriptor such that at least a portion of information in the polyamino acid descriptor is lost in the latent descriptor; and determining, based at least in part on the latent descriptor, the biological state associated with the polyamino acid descriptor.

In some embodiments, the latent descriptor comprises a plurality of parameters for a posterior distribution. In some embodiments, the posterior distribution is tighter than a normal distribution. In some embodiments, the prior distribution comprises a higher kurtosis than a normal distribution. In some embodiments, the posterior distribution is a Laplace distribution. In some embodiments, the generating in (b) is performed at least in part by using an encoder. In some embodiments, the encoder is trained, while coupled with a decoder, to reconstruct a plurality of polyamino acid descriptors in a training dataset. In some embodiments, during training, the decoder outputs a plurality of parameters for a probability distribution. In some embodiments, the probability distribution is a zero inflated distribution. In some embodiments, the zero inflated distribution is a zero inflated negative binomial distribution. In some embodiments, the polyamino acid descriptor comprises at least 100 dimensions. In some embodiments, the latent descriptor comprises at most about 50% of the number of dimensions in the polyamino acid descriptor. In some embodiments, the polyamino acid descriptor comprises more zero values than non-zero values. In some embodiments, at least about 10% of values in the polyamino acid descriptor are zero. In some embodiments, the given assay method comprises contacting a plurality of biomolecules with a given surface. In some embodiments, the given surface is a surface of a particle. In some embodiments, the given assay method comprises (i) performing mass spectrometry on cleaved derivatives of the plurality of biomolecules to obtain a plurality of peptide spectral signals and (ii) processing the plurality of peptide spectral signals to obtain a plurality of peptide identifications, wherein the plurality of polyamino acid descriptors comprises the plurality of peptide identifications. In some embodiments, the given assay method comprises (i) performing mass spectrometry on cleaved derivatives of the plurality of biomolecules to obtain a plurality peptide spectral signals (ii) processing the plurality of peptide signals to obtain a plurality of peptide identifications, and (iii) processing the plurality of peptide identifications to obtain a plurality of intensities for plurality of protein or protein group identification, wherein the plurality of polyamino acid descriptors comprises the plurality of protein or protein group identifications. In some embodiments, the method further comprises determining at least one decision boundary in the latent space, wherein the at least one decision boundary distinguishes between a first polyamino acid descriptor associated with a first biological state and a second polyamino acid descriptor associated with a second biological state. In some embodiments, the method further comprises determining at least one biomarker associated with a biological state in the first polyamino acid descriptor, based at least partially on the at least one decision boundary. In some embodiments, the method further comprises determining at least one direction in the latent space, wherein the at least one direction is associated with a change in a first polyamino acid descriptor that is correlated with a change in a biological state. In some embodiments, the method further comprises determining at least one biomarker associated with a biological state in the polyamino acid descriptor, based at least partially on the at least one direction. In some embodiments, the at least one biomarker comprises a plurality of proteins or protein groups. In some embodiments, the at least one biomarker comprises a plurality of peptides. In some embodiments, the determining the biological state is performed using a machine learning model. In some embodiments, the machine learning model is selected from the group consisting of a support vector machine, a random forest, and a multi-layer perceptron. In some embodiments, the determining the biological state is characterized by an area under a receiving operating characteristic curve of at least 0.6. In some embodiments, the determining the biological state is characterized by an area under a receiving operating characteristic curve of at least 0.8. In some embodiments, the determining the biological state is characterized by an area under a receiving operating characteristic curve of at least 0.9. In some embodiments, the determining the biological state is characterized by an area under a receiving operating characteristic curve of at least 0.95. In some embodiments, the determining the biological state is characterized by an area under a receiving operating characteristic curve of at least 0.99.

Another aspect of the present disclosure provides a computer-implemented method, comprising implementing any one of the methods for determining a biological state associated with a polyamino acid descriptor described herein.

Another aspect of the present disclosure provides a computer program product comprising a computer-readable medium having computer-executable code encoded therein, the computer-executable code adapted to be executed to implement any one of the methods for determining a biological state associated with a polyamino acid descriptor described herein.

Another aspect of the present disclosure provides a non-transitory computer-readable storage media encoded with a computer program including instructions executable by one or more processors to determine a biological state using any one of the methods described herein.

Another aspect of the present disclosure provides a computer-implemented system comprising: a digital processing device comprising: at least one processor, an operating system configured to perform executable instructions, a memory, and a computer program including instructions executable by the digital processing device to determine a biological state using any one of the methods described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C schematically illustrates an example neural network architecture, in accordance with some embodiments.

FIGS. 2A-2B illustrate PCA projection of proteomic data, in accordance with some embodiments.

FIGS. 3A-3B show receiver operating characteristic curves of classifiers, in accordance with some embodiments.

FIGS. 4A-4B show the distribution of identified protein group counts, in accordance with some embodiments.

FIG. 5 shows differentially expressed protein groups across the cohort, in accordance with some embodiments.

FIG. 6 shows hierarchical clustering on the differentially expressed protein intensities, in accordance with some embodiments.

FIGS. 7A-7F show functional enrichment of differentially expressed proteins, in accordance with some embodiments.

FIG. 8 shows a computer system, in accordance with some embodiments.

### DETAILED DESCRIPTION

Disclosed herein, in one aspect, are methods and systems for generating embeddings of biomolecular data. In some embodiments, the present disclosure provides for methods for training an algorithm (e.g., a machine learning algorithm, such as a neural network) for generating encodings of biological data. Direct analysis of biological data (e.g., feature intensities from genomic, transcriptomic, or proteomic analyses) may present challenges when used to train algorithms. for clinical or for research tasks. Feature intensities can be reported as sparse vectors (e.g., many entries are zero or vanishingly small) in a high-dimensional space (e.g., comprising at least 100, 1,000, or 10,000 dimensions), requiring trained algorithms to learn many parameters. Additionally, many dimensions may contain redundant, unnecessary information, or noise which can inflate the data, thereby leading to overfitting and hampering interpretability. To overcome one or more of these drawbacks, methods and systems are disclosed herein which are able to generate reduced-dimension representation of biological data (such as feature intensities) from input biomolecule descriptors. The reduced-dimension representations may permit robust, noise free identification of the biomolecules present in a sample.

The biomolecule embeddings may further permit identification of novel biomarkers based on deep plasma biomolecule profiling with multi-surface panel platform. The methods and systems described herein can permit superior biomolecule-based biomarker (e.g., for cancer, such as non-small cell lung cancer) discovery using a biomolecule profiling platform of panels of particle types disclosed herein for quantification of plasma biomolecules. Surfaces (e.g., surfaces of nanoparticles) can specifically and reproducibly interrogate subsets of biomolecules from biofluids for biomolecule profiling, efficiently and effectively. When data derived from these biomolecule profiling experiments are encoded in the reduced representations described herein, they can be processed to determine new associations between biomolecule-based biomarkers and biological categories of biological scales, such as types of disease or extent of disease progression.

### Methods and Systems for Biomolecule Embeddings

In some aspects, the present disclosure provides methods for training a neural network to generate an embedding of a biomolecule descriptor (e.g., polyamino acid descriptor). The methods may comprise an operation of providing a neural network comprising an input layer. The methods may comprise an operation of providing a neural network comprising a latent layer connected to the input layer. The methods may comprise an operation of providing a neural network comprising an output layer connected to the latent layer. The input layer can be configured to receive one or more input biomolecule (e.g., polyamino acid) descriptors. The polyamino acid descriptor(s) can comprise a (e.g., numerical) value characterizing a polyamino acid. In some cases, the polyamino acid descriptor comprises a plurality of numerical values, such as a scalars, vectors, matrices, or higher-order tensors. In some cases, the value is not numerical. For example, the value may comprise categorical data. In some cases, non-numerical (e.g., categorical) data may be converted to a numerical representation (e.g., one-hot encodings) for use with the methods and systems of the disclosure.

The biomolecule descriptor can comprise biological data (including proteomic, genomic, transcriptomic, or proteogenomic data) as described elsewhere herein. In some cases, the biomolecule descriptor comprises one or more values derived from an assay. In some cases, the biomolecule descriptor comprises one or more biomolecule (e.g., peptide, protein, or protein groups) identifications. In some cases, the biomolecule descriptor comprises one or more measured intensities (e.g., feature intensities). The measured intensities can be obtained using a variety of methods and/or instrumentation. The measured intensities can comprise mass spectrometry (MS) intensities. The MS intensities can comprise peptide intensities, protein group intensities, or both. The MS intensities can comprise small molecule intensities. The MS intensities can be based on data-independent acquisition (DIA) MS, data-dependent acquisition (DDA) MS, or both. The MS intensities can be based on liquid-chromatography tandem mass spectrometry (LC-MS/MS). In some cases, the assay comprises contacting a plurality of biomolecules (e.g., comprised in a sample or a derivative thereof) with a surface to bind one or more of the plurality of biomolecules. In such cases, the assay can comprise performing mass spectrometry on (e.g., a subset) of the plurality of biomolecules. The performing mass spectrometry may comprise performing mass spectrometry on a plurality of derivatives (e.g., proteolytically or chemically cleaved peptides) of the one or more biomolecules. In such cases, the biomolecule descriptors may comprise a plurality of derivative (e.g., peptide) spectral signals or identifications (e.g., peptide identifications) derived therefrom. In some cases, the biomolecules comprise a plurality of feature intensities for protein or protein group identifications based on the peptide. identifications. In some cases, the surface is a surface of a particle. In some cases, the particle is a nanoparticle.

Those skilled in the art will understand that the biomolecule descriptor may comprise various numbers of dimensions. The biomolecule descriptor may comprise a number of dimensions. The biomolecule descriptor may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 50,000, or 100,000, or more dimensions. In some cases, the biomolecule descriptor comprises no more than about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or 1 dimension.

The latent layer may be configured to output a latent descriptor (e.g., latent representation or latent embedding) of the biomolecule descriptor. The latent descriptor can comprise fewer dimensions than the input descriptor. In some cases, the latent descriptor can comprise more dimensions than the input descriptor. In some cases, the latent descriptor can comprise a reduced dimensionality compared to the biomolecule descriptor by a certain proportion. In some cases, the latent descriptor can comprise reduced dimensionality compared to the biomolecule descriptor by at least 1, 2, 3, 4, 5, 6, 7, 8, 9, 10, 20, 30, 40, 50, 60, 70, 80, 90, 91, 92, 93, 94, 95, 96, 97, 98, or 99 percent. In some cases, the latent descriptor can comprise reduced dimensionality compared to the biomolecule descriptor by at most 1, 2, 3, 4, 5, 6, 7, 8, 9, 10, 20, 30, 40, 50, 60, 70, 80, 90, 91, 92, 93, 94, 95, 96, 97, 98, or 99 percent. In some cases, the latent descriptor can comprise reduced dimensionality compared to the biomolecule descriptor by a certain number of dimensions. In some cases, the latent descriptor can comprise reduced dimensionality compared to the biomolecule descriptor by at least about 1, 2, 5, 10, 20, 30, 40, 50, 100, 200, 500, 1,000, 2,000, 5,000, or 10,000 or more dimensions In some cases, the latent descriptor can comprise reduced dimensionality compared to the biomolecule descriptor by at most about 10,000, 5,000, 2,000, 1,000, 500, 200, 100, 50, 40, 30, 20, 10, 5, 2, or 1 dimension.

The biomolecule descriptor may comprise a noise. As described elsewhere herein, biomolecule descriptors may comprise sparse data of high dimensionality. Accordingly, methods and systems as described herein may be configured to reduce a noise in a latent description, relative to an input biomolecule descriptor. The reduction in noise may be due to the neural network learning which dimensions comprise more significant information.

In some cases, the plurality of polyamino acid descriptors comprise sparse data. For example, the plurality of polyamino acid descriptors may comprise a certain proportion of zero values (e.g., in a vector). In some cases, the plurality of biomolecule descriptors comprises more zero values than non-zero values. In some cases, the plurality of biomolecule descriptors comprises at least about 1%, 2%, 3%, 4%, 5%, 6%, 7%, 8%, 9%, 10%, 15%, 20%, 25%, 30%, 35%, 40%, 45%, 50%, 56%, 60%, 65%, 70%, 75%, 80%, 85%, 90%, or 95%, or more zero values. In some cases, the plurality of polyamino acid comprises at most about 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1%, or less zero values.

In some cases, (e.g., in the case of a variational autoencoder as described herein below) the latent descriptor may comprise a probability distribution or a plurality of probability distributions. The neural network may be trained to learn a probability distribution over the input biomolecule descriptors. In such cases, the latent descriptor (e.g., output of a latent layer) may comprise a parameter or plurality of parameters characterizing the trained probability distribution. In some cases, the latent descriptor comprises a parameter of a posterior distribution. In some cases, the latent descriptor comprises a plurality of parameters of a plurality of posterior distributions. In some cases, the latent descriptor comprises a plurality of parameters of a plurality of posterior distributions.

The methods as described herein may comprise an operation of providing training data comprising a plurality of biomolecule descriptors. In some cases, the training data comprises paired inputs and target outputs. In some cases, the training data comprises only inputs, such as when the neural network comprises an autoencoder (e.g., variational autoencoder) or other architecture configured to reconstruct the input data through a latent representation. The training data may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 50,000, 100,000, 1,000,000, 10,000,000, or more data. In some cases, the biomolecule descriptor comprises no more than about 10,000,000, 1,000,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or 1 data. In some cases, the training data comprise numerical values. In some cases, the training data comprise categorical values.

The methods may further comprise an operation of training the trained algorithm (e.g., neural network). The training operation may comprise inputting the training data (e.g., biomolecule descriptors) into the input layer of the neural network. The training operation may further comprise outputting a (e.g., plurality of) latent descriptor and a plurality of reconstructions at the output layer. Based on a measured difference between at least a subset of the input data and the reconstructions, one or more parameters of the neural network may be adjusted. The difference or deviation between the input and the reconstruction may be measured by a loss function. As a result of the training, the neural network may learn a latent space for encoding input data. The latent space may comprise a reduced dimension relative to the input data. When the inputs are embedded in the latent space, they may comprise a reduced noise compared to the original inputs.

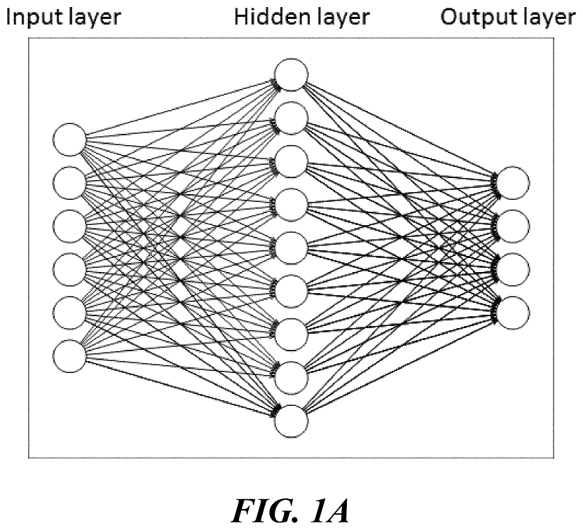

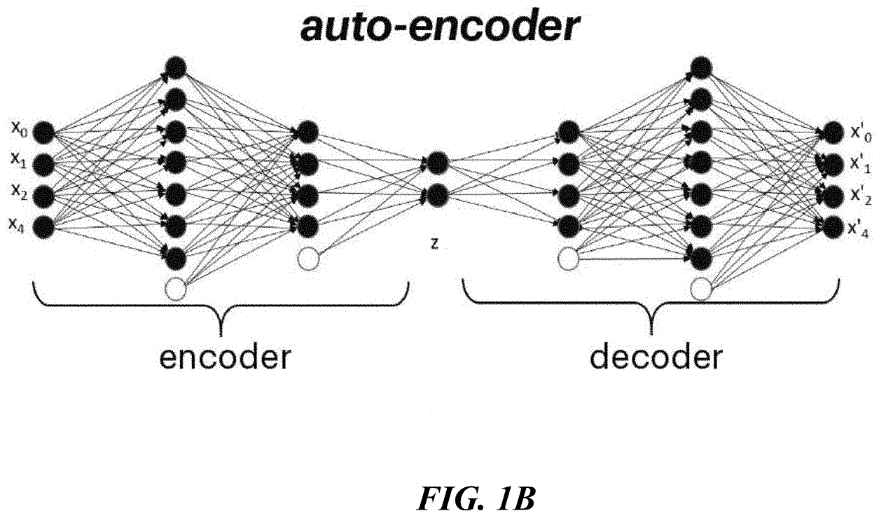

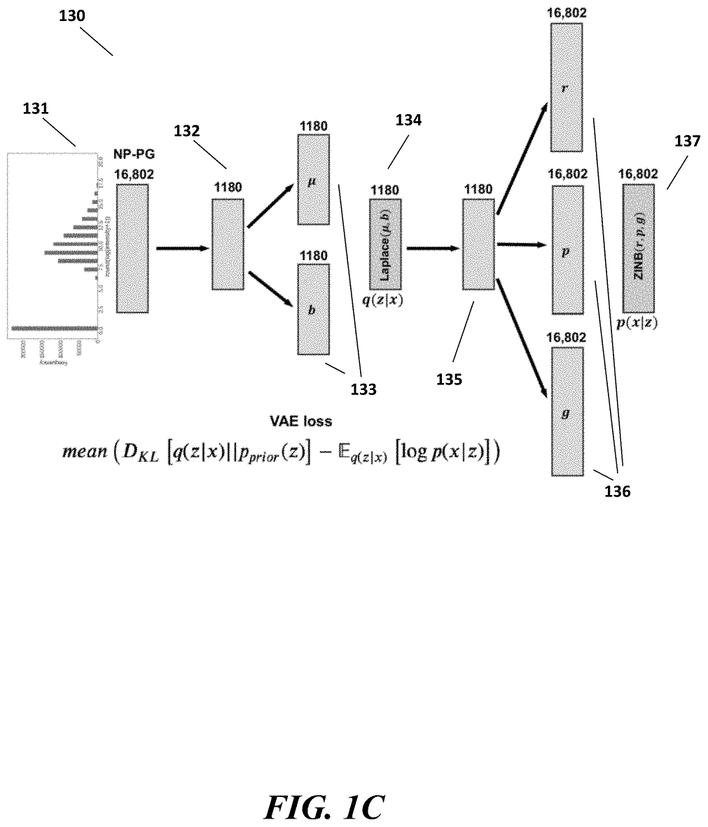

Example neural networks for use with the methods and systems of the present disclosure are illustrated in FIG. 1A. Neural networks generally comprise an interconnected group of nodes organized into multiple layers of nodes. For example, the neural network architecture may comprise at least an input layer, one or more hidden layers, and an output layer (FIG. 1A). The neural network may comprise any total number of layers, and any number of hidden layers, where the hidden layers function as trainable feature extractors that allow mapping of a set of input data to a preferred output value or set of output values. Each layer of the neural network can comprise a number of nodes (or neurons). A node can receive input that comes either directly from the input data (e.g., biomolecule descriptor data) or the output of nodes in previous layers, and performs a specific operation, e.g., a summation operation. In some cases, a connection from an input to a node is associated with a weight (or weighting factor) and optionally a bias. In some cases, the output of a neuron may be gated using a threshold or activation function which may be a linear or non-linear function. The activation function may be, for example, a rectified linear unit (ReLU) activation function or other function such as a saturating hyperbolic tangent, identity, binary step, logistic, arctan, softsign, parametric rectified linear unit, exponential linear unit, softPlus, bent identity, softExponential, Sinusoid, Sinc, Gaussian, or sigmoid function, or any combination thereof.

The weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) that the neural network computes are consistent with the examples included in the training data set. The adjustable parameters of the model may be obtained from a back propagation neural network training process.

Other specific types of deep machine learning algorithms for use with the methods and systems disclosed herein include, e.g., autoencoders. An autoencoder can refer to a neural network used for unsupervised or self-supervised mapping of input data to an output value. FIG. 1B illustrates a basic architecture of an example autoencoder. An autoencoder generally comprises an encoder which generates a representation (e.g., encoding) of an input and a decoder which reconstructs the input from the representation. As illustrated in FIG. 1B, the encoder may comprise a plurality of encoding layers. Each encoding layer may comprise a plurality of nodes, each of which is associated with a numerical operator, an activation function, a bias, and/or a plurality of numerical weights. Similarly, the decoder may further comprise a plurality of decoding layers. Each decoding layer may comprise a plurality of nodes, each of which is also associated with a numerical operator, an activation function, a bias, and/or a plurality of numerical weights. Autoencoders can permit dimensionality reduction by transforming the input into a representation comprising fewer dimensions than the input (e.g., as illustrated in FIG. 1B). Dimensionality reduction may be performed, for example, for the purpose of feature selection (e.g., selection of the most relevant subset of biomolecule descriptors present in a) or feature extraction (e.g., transformation of biomolecule descriptor data in an original, multi-dimensional space to a space of fewer dimensions). The neural network may be configured to distill a dataset having high dimensionality (e.g., biomolecule descriptors) into a reduced set of numerical values that still represents the essential features of the input dataset while eliminating redundancy or noise.

Examples of autoencoders for use with the presently disclosed methods and systems include, but are not limited to, sparse autoencoders, stacked autoencoders, denoising autoencoders, contractive autoencoders, variational autoencoders, or any combination thereof. Variational autoencoders (VAEs) can refer to autoencoder models that use the basic autoencoder architecture of an encoder and decoder, while using a variational approach for latent representation learning and are generally trained at a task of reconstructing their own input. Rather than encode and decode a single latent space representation of an input, VAEs can encode and decode inputs and encodings using conditional probability distributions. VAEs may be trained by adjusting the parameters of these conditional probability distributions to optimize a training or loss function. The loss function may comprise a reconstruction loss which assesses how close in expectation the reconstruction of an input is to the original input. The loss function may additionally or alternatively comprise a Kullback-Leibler divergence (KL divergence). The KL divergence may measure how well a certain parameterization (e.g., of a latent distribution) approximates a target distribution. In some cases, the loss function may comprise a reconstruction loss and a KL divergence between a target latent space distribution and its parameterization by one or more posterior distributions.

In an example, a VAE comprises a ϕ-parameterized encoder which maps an input x∈X to a latent space Z using a distribution q_{ϕ}(z|x). Samples from q_{ϕ}(z|x) are then reconstructed using a decoder p_{θ}(x|z). The model assumes the existence of some prior distribution on the latent representations, p(z). ϕ and θ represent the learnable parameters of the encoder and decoder and are generally parameters of the distributions q(z|x) and p(x|z). The distribution(s) p_{θ}(z|x) may be referred to as the posterior distribution(s) herein. A latent layer of the VAE may be configured to output a plurality of parameters for the posterior distribution(s). The plurality of parameters may comprise one or more sets of parameters corresponding to dimensions of the latent space.

The prior distribution and the encoder and decoder distributions may comprise any probability distribution. Examples of probability distributions include, but are not limited to, the uniform distribution, the Bernoulli distribution, the Rademacher distribution, the binomial distribution, the beta-binomial distribution, the degenerate distribution, the discrete uniform distribution, the hypergeometric distribution, the Poisson binomial distribution, the Fisher's noncentral hypergeometric distribution, Wallenius' noncentral hypergeometric distribution, the beta negative binomial distribution, the Boltzmann distribution, the Gibbs distribution, the Maxwell-Boltzmann distribution, the Borel distribution, the extended negative binomial distribution, the extended hypergeometric distribution, the generalized log-series distribution, the geometric distribution, the logarithmic (series) distribution, the negative binomial distribution, the zero-inflated negative binomial distribution, the discrete compound Poisson distribution, the parabolic fractal distribution, the Poisson distribution, the Conway-Maxwell-Poisson distribution, the zero-truncated Poisson distribution, the zero-inflated Poisson distribution, the Polya-Eggenberger distribution, the Skellam distribution, the skew elliptical distribution, the Yule-Simon distribution, the zeta distribution, the Zipf distribution, the Behrens-Fisher distribution, the Cauchy distribution, the Chernoffs distribution, the Exponentially modified Gaussian distribution, the Fisher's z-distribution, the skewed generalized t-distribution, the generalized logistic distribution, the generalized normal distribution, the geometric stable distribution, the Gumbel distribution, the Holtsmark distribution, the hyperbolic distribution, the hyperbolic secant distribution, the Johnson SU distribution, the Landau distribution, the Laplace distribution, the Levy skew alpha-stable distribution, the Linnik distribution, the logistic distribution, the map-Airy distribution, the normal distribution, the normal-exponential-gamma distribution, the normal-inverse Gaussian distribution, the Pearson Type IV distribution, the skew normal distribution, the Student's t-distribution, the noncentral t-distribution, the skew t-distribution, the Champernowne distribution, the type-1 Gumbel distribution, the Tracy-Widom distribution, the Voigt distribution, the beta prime distribution, the Birnbaum-Saunders distribution, the chi distribution, the noncentral chi distribution, the chi-squared distribution, the inverse-chi-squared distribution, the noncentral chi-squared distribution, the scaled inverse chi-squared distribution, the Dagum distribution, the exponential distribution, the exponential-logarithmic distribution, the F-distribution, the noncentral F-distribution, the folded normal distribution, the Fréchet distribution, the Gamma distribution, the Erlang distribution, the inverse-gamma distribution, the generalized gamma distribution, the generalized Pareto distribution, the Gamma/Gompertz distribution, the Gompertz distribution, the half-normal distribution, the Hotelling's T-squared distribution, the inverse Gaussian distribution, the Levy distribution, the log-Cauchy distribution, the log-Laplace distribution, the log-logistic distribution, the Lomax distribution, the Mittag-Leffler distribution, the Nakagami distribution, the Pareto distribution, the Pearson Type III distribution, the phase-type distribution, the phased bi-exponential distribution, the phased bi-Weibull distribution, the Rayleigh distribution, the Rayleigh mixture distribution, the Rice distribution, the shifted Gompertz distribution, the type-2 Gumbel distribution, and the Weibull distribution.

In some cases, prior or posterior distribution(s) may be chosen based on certain properties of the distributions. In some cases, a probability distribution may be chosen based at least in part on an underlying process the probability distribution is used to model or represent. In some cases, the underlying process being modeled has a finite number of possible outcomes for which a discrete distribution is chosen. In some cases, the underlying process being modeled can take on any value within an (e.g., continuous) interval for which a continuous probability distribution is chosen. In some cases, the underlying process may have certain features which should be reflected in a corresponding probability distribution. For example, for modeling sparse data (e.g., comprising many zeros) or data which is expected to generally be sparse, a zero-inflated probability distribution may be used as the prior distribution to model sparsity and encourage sparsity in latent representations of the data (e.g., generated by an autoencoder, such as a variational autoencoder). In some cases, a zero-inflated distribution comprises a zero-inflated Poisson distribution. In some cases, a zero-inflated distribution comprises a zero-inflated negative binomial distribution.

In an example,

In some cases, a probability distribution is chosen based in part on a (e.g., relative) value of a moment or cumulant of the probability distribution. The moment cumulant can be a raw, central, or standardized moment. For example, a probability distribution may be chosen based on its mean, variance, skewness, kurtosis, excess kurtosis, hyperskewness, hypertailedness, or any combination thereof. In some cases, a probability distribution comprises a certain mean. In some cases, a probability distribution comprises a certain variance. In some cases, a probability distribution comprises a certain skewness. In some cases, a probability distribution comprises a certain kurtosis. In some cases, a probability distribution comprises a certain excess kurtosis. In some cases, a probability distribution comprises a certain hyperskewness. In some cases, a probability distribution comprises a mean greater than a reference (e.g., relative to a Gaussian) distribution. In some cases, a probability distribution comprises a variance greater than a reference (e.g., relative to a Gaussian) distribution. In some cases, a probability distribution comprises a skewness greater than a reference (e.g., relative to a Gaussian) distribution. In some cases, a probability distribution comprises a kurtosis greater than a reference (e.g., relative to a Gaussian) distribution. In some cases, a probability distribution comprises an excess kurtosis greater than a reference (e.g., relative to a Gaussian) distribution. In some cases, a probability distribution comprises a hyperskewness greater than a reference (e.g., relative to a Gaussian) distribution. In some cases, a probability distribution comprises a hypertailedness greater than a reference (e.g., relative to a Gaussian) distribution. In some cases, a probability distribution comprises a mean less than a reference (e.g., relative to a Gaussian) distribution. In some cases, a probability distribution comprises a variance less than a reference (e.g., relative to a Gaussian) distribution. In some cases, a probability distribution comprises a skewness less than a reference (e.g., relative to a Gaussian) distribution. In some cases, a probability distribution comprises a kurtosis less than a reference (e.g., relative to a Gaussian) distribution. In some cases, a probability distribution comprises an excess kurtosis less than a reference (e.g., relative to a Gaussian) distribution. In some cases, a probability distribution comprises a hyperskewness less than a reference (e.g., relative to a Gaussian) distribution. In some cases, a probability distribution comprises a hypertailedness less than a reference (e.g., relative to a Gaussian) distribution. In an example, in order to capture sparsity in the input dataset, a relatively narrow (e.g., narrower than a reference distribution, such as a Gaussian distribution) may be selected as the posterior distribution(s). Alternatively, or additionally, the posterior distribution(s) may comprise a distribution with a relatively higher kurtosis (e.g., relative to a reference distribution, such as a Gaussian distribution). A distribution with a relatively higher kurtosis (e.g., as compared to a Gaussian distribution) has a relatively greater proportion of its density or mass distributed in the tails of the distribution. Accordingly, such distributions can be suitable for modeling processes which generate a relatively greater number of outliers. In an example, a posterior distribution comprises a Laplacian distribution.

FIG. 1C illustrates an example trained neural network 130 in accordance with the methods and systems of the present disclosure. The neural network 130 illustrated in FIG. 1C comprises a variational autoencoder (VAE) architecture configured to learn an approximation of a distribution of biomolecule descriptors 131 (illustrated in FIG. 1C as a distribution of feature intensities from a proteomic analysis of a plurality of biological samples). The distribution of biomolecule descriptors 131 comprises a plurality of polyamino acid descriptors comprising feature intensities (e.g., as measured by mass spectrometry operations as described elsewhere herein) as measured across biological samples. Due to the large number of samples and protein groups detected, the data exists in a high-dimensional space (e.g., 16,802 dimensions). Further, the data are relatively sparse (e.g., comprise a certain proportion of zero entries, such as at least about 10%, 20%, 30%, 40%, 50%, 60%, 70%, 80%, 90%, 95%, or 99%, or more zero entries). Neural network 130 further comprises a fully connected encoder 132 which is configured to output a plurality of posterior distribution parameters 133. As illustrated in FIG. 1C, the posterior distributions 134 encoding the input data are Laplacian distributions parameterized by location parameters μ and scale parameters b. The example illustrated in FIG. 1C shows a reduction in dimensionality of about 93% (from 16,802 dimensions to 1,180 dimensions), though other reductions, such as by at least about 10%, 20%, 30%, 40%, 50%, 60%, 70%, 80%, 90%, 95%, or 99%, or more, or at most about 99%, 95%, 90%, 80%, 70%, 60%, 50%, 40%, 30%, 20%, or 10%, or less, are possible. The VAE further comprises a fully-connected decoder 135 which is configured to decode the latent representations of the encoder back into the input space. The decoder outputs parameters 136 (r, p, g) which are the parameters of a zero-inflated negative binomial distribution 137.

In the example illustrated in FIG. 1C, the posterior distributions are selected as Laplacian distributions. Relative to a reference (e.g., Gaussian) distributions, Laplacian distributions are narrower can thus better model sparsity in the original data. Additionally, Laplacian distributions are leptokurtic relative to certain reference (e.g., Gaussian) distributions and can thus better model data which show a relatively higher proportion of outliers. In other cases, another posterior distribution or distributions can be employed which are relatively narrower (e.g., relative to a reference distribution, such as a Gaussian distribution) to model sparse data. In other cases, another posterior distribution or distributions can be employed which are relatively leptokurtotic (e.g., relative to a reference distribution, such as a Gaussian distribution) to model sparse data. The example illustrated in FIG. 1C further employs a zero-inflated negative binomial (ZINB) distribution as the prior distribution. In this example, the illustrated VAE is being trained to sparse data (e.g., data comprising a relatively high number of zero values). Accordingly, a zero-inflated distribution, such as a ZINB distribution, can better model the data than a corresponding distribution which is not zero-inflated (e.g., negative binomial distribution or Poisson distribution). In other cases, another zero-inflated distribution is employed to model sparse data as described herein.

FIG. 1C further illustrates a representative loss function for training neural network 130. The loss function is

mean(D_{KL}[q(z|x)∥pₚᵣᵢₒᵣ(z)]−E_{q(z|x)}[log p(x|z)])

where D_{KL}[q(z|x)∥pₚᵣᵢₒᵣ(z)] is the KL divergence between the prior distribution and the posterior distributions and E_{q(z|x)}[log p(x|z)] is the reconstruction loss which measures the likelihood of the predicted reconstruction.

### Classifying Biological States

An aspect of the present disclosure further provides methods and systems for classifying biological states. The biological state may be classified on the basis of data (e.g., proteomic, genomic, proteogenomic, such as biomolecule descriptors) derived from a subject or a sample form the subject. In some cases, the data comprise polyamino acid descriptors. In some cases, the classification may be performed based on a transformation or encoding of the polyamino acid descriptors as described herein (e.g., a latent descriptor or representation). In some cases, the biological state is associated with a polyamino acid descriptor. In some cases, methods and systems as described herein comprise an operation of associating a biomolecule descriptor with a biological state. In some cases, the associating comprises associating a latent representation of a biomolecule descriptor with the biological state. In some cases, the associating comprises associating a plurality of biomolecule descriptors with the biological state. In some cases, the associating comprises associating a plurality of biomolecule descriptors with a plurality of biological states.

The association may be based on a latent descriptor (e.g., latent embedding or representation) of the biomolecule descriptor. The latent descriptor may be generated by a trained algorithm (e.g., neural network, such as an encoder) as described herein. In some cases, the latent descriptor may comprise a reduced dimension relative to the input biomolecule descriptor, such that some information in the latent descriptor is lost relative to the input biomolecule descriptor. In some cases, the latent descriptor may comprise a reduced dimension relative to the input biomolecule descriptor such that noise is reduced in the latent descriptor relative to the input biomolecule descriptor.

In some cases, methods and systems as described herein comprise determining one or more biomarkers associated with a biological state. The one or more biomarkers may comprise one or more polyamino acid descriptors. In some cases, the one or more biomarkers comprise one or more peptides. In some cases, the one or more biomarkers comprise one or more proteins or protein groups.

In some cases, methods and systems as described herein may comprise classifying a set of latent descriptors (e.g., biomolecule descriptors) associated with a biological state (e.g., a first biological state). From the set of latent descriptors, a corresponding (e.g., first set) of biomolecule descriptors may be generated and associated with the biological state. In some cases, the set of biomolecule descriptors may comprise at least one biomolecule descriptor. In some cases, the set of biomolecule descriptors comprises zero biomolecule descriptors (e.g., is associated with an absence of biomolecule descriptors). In some cases, the set of biomolecule descriptors is obtained by decoding (e.g., passing through a decoder, such as a decoder of an autoencoder) the set of latent descriptors. In some cases, the (e.g., decoded) biomolecule descriptors may be used to identify a set of biomarkers associated with the biological state. In some cases, the set of biomarkers comprises a peptide. In some cases, the set of biomarkers comprises a protein. In some cases, the set of biomarkers comprises a protein group. In some cases, the methods and systems may comprise classifying another set of latent descriptors (e.g., a second set), with a second biological state.

The biomarkers described herein can be used individually or in combination in diagnostic tests to assess disease state or status of a subject. Disease status can include the presence or absence of a particular disease. In some cases, the disease state or condition comprises a cancer (e.g., breast, lung, liver, kidney, gastric, or colorectal cancer), a particular stage of cancer, a tissue of origin of a cancer, a particular subtype of cancer, a predicted response to a therapeutic intervention for a cancer, a risk level (e.g., high risk, low risk) for a particular cancer or subtype of cancer, or a predicted long-term outcome of a cancer (e.g., distant metastasis, biochemical recurrence, partial response, complete response, overall survival, cancer-specific survival, progression free survival, five year survival, disease free survival, death), or any combination thereof. Disease (e.g., cancer) status may also include monitoring the course of the disease, for example, monitoring disease progression, subtype (including subtype selection, switching or conversion) or determining the level of residual tumor burden. Based on the determined disease status of a subject, additional procedures may be indicated, including, for example, additional diagnostic tests or therapeutic procedures.

Non-limiting examples of tumors and associated cancers that may be detected, diagnosed, prognosed, and the like by methods and systems as described herein include acoustic neuroma, acute lymphoblastic leukemia, acute myeloid leukemia, adenocarcinoma, adrenocortical carcinoma, AIDS-related cancers, AIDS-related lymphoma, anal cancer, angiosarcoma, appendix cancer, astrocytoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, bronchogenic carcinoma, cerebellar astrocytoma, cervical cancer, childhood cancers, chondrosarcoma, chordoma, choriocarcinoma, chronic lymphocytic leukemia, chronic myelogenous leukemia, chronic myeloproliferative disorders, colon cancer, colon carcinoma, craniopharyngioma, cutaneous T-cell lymphoma, cystadenocarcinoma, desmoplastic small round cell tumor, embryonal carcinoma, endocrine system carcinomas, endometrial cancer, endotheliosarcoma, ependymoma, epithelial carcinoma, esophageal cancer, Ewing's sarcoma, fibrosarcoma, germ cell tumors, gallbladder cancer, gastric cancer, gastrointestinal carcinoid tumor, gastrointestinal stromal tumor, gastrointestinal system carcinomas, genitourinary system carcinomas, gliomas, hairy cell leukemia, head and neck cancer, heart cancer, hemangioblastoma, hepatocellular (liver) cancer, Hodgkin lymphoma, Hypopharyngeal cancer, intraocular melanoma, islet cell carcinoma, Kaposi sarcoma, kidney cancer, laryngeal cancer, leiomyosarcoma, lip and oral cavity cancer, liposarcoma, liver cancer, lung cancers, such as non-small cell (NSC) and small cell (SC) lung cancer (LC), lung carcinoma, lymphangiosarcoma, lymphangioendotheliosarcoma, lymphomas, leukemias, macroglobulinemia, malignant fibrous histiocytoma of bone/osteosarcoma, medulloblastoma, medullary carcinoma, melanomas, meningioma, mesothelioma, metastatic squamous neck cancer with occult primary, mouth cancer, multiple endocrine neoplasia syndrome, myelodysplastic syndromes, myeloid leukemia, myxosarcoma, nasal cavity and paranasal sinus cancer, nasopharyngeal carcinoma, neuroblastoma, non-Hodgkin lymphoma, non-small cell lung cancer, oligodendroma, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, papillary adenocarcinoma, papillary carcinoma, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, sebaceous gland carcinoma, seminoma, skin cancers, skin carcinoma merkel cell, small intestine cancer, soft tissue sarcoma, squamous cell carcinoma, gastric cancer, sweat gland carcinoma, synovioma, T-cell lymphoma, testicular tumor, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, Wilms tumor, or combinations thereof. The tumors or cancers may be associated with various types of organs. Non-limiting examples of organs may include brain, breast, colon, liver, lung, kidney, prostate, ovary, spleen, lymph node (including tonsil), thyroid, pancreas, heart, skeletal muscle, intestine, larynx, esophagus, stomach, small intestine, or combinations thereof.

The power of a diagnostic test to correctly predict disease status can be measured in terms of the accuracy of the assay, the sensitivity of the assay, the specificity of the assay, the positive predictive value (PPV) of the assay, the negative predictive value (NPV) of the assay, or the area under a Receiver Operating Characteristic (ROC) curve (AUC). As used herein, accuracy is a measure of the fraction of misclassified samples. Accuracy may be calculated as the total number of correctly classified samples divided by the total number of samples, e.g., in a test population. Sensitivity is a measure of the “true positives” that are predicted by a test to be positive and may be calculated as the number of correctly identified breast cancer samples divided by the total number of breast cancer samples. Specificity is a measure of the “true negatives” that are predicted by a test to be negative and may be calculated as the number of correctly identified normal samples divided by the total number of normal samples. AUC is a measure of the area under a Receiver Operating Characteristic curve, which is a plot of sensitivity vs. the false positive rate (1−specificity). The greater the AUC, the more powerful the predictive value of the test. Other useful measures of the utility of a test include the “positive predictive value,” which is the percentage of actual positives who test as positives, and the “negative predictive value,” which is the percentage of actual negatives who test as negatives. In some embodiments, the level of one or more biomarkers in samples derived from subjects having different cancer statuses show a statistically significant difference of at least p=0.05, e.g., p=0.05, p=0.01, p=0.005, p=0.001, etc. relative to normal subjects, as determined relative to a suitable control. In some embodiments, diagnostic tests that use biomolecule descriptors or their representations (e.g., latent descriptors) as described herein individually or in combination show an accuracy of at least about 75%, e.g., an accuracy of at least about 75%, about 80%, about 85%, about 90%, about 95%, about 97%, about 99%, about 99.9% or about 100%. In other embodiments, diagnostic tests that use biomolecule descriptors or their representations described herein individually or in combination show a specificity of at least about 75%, e.g., a specificity of at least about 75%, about 80%, about 85%, about 90%, about 95%, about 97%, about 99%, about 99.9%, or about 100%. In some embodiments, diagnostic tests that use biomolecule descriptors or their representations described herein individually or in combination show a sensitivity of at least about 75%, e.g., a sensitivity of at least about 75%, about 80%, about 85%, about 90%, about 95%, about 97%, about 99%, about 99.9%, or about 100%. In other embodiments, diagnostic tests that use biomarkers described herein individually or in combination can show a specificity and sensitivity of at least about 75% each, e.g., a specificity and sensitivity of at least about 75%, about 80%, about 85%, about 90%, about 95%, about 97%, about 99% or about 100% (for example, a specificity of at least about 80% and sensitivity of at least about 80%, or for example, a specificity of at least about 80% and sensitivity of at least about 95%).

By way of nonlimiting example, each biomarker listed in Tables 1 is differentially present in biological samples derived from subjects having NSCLC as compared with normal subjects, and thus each may be used individually or in any combination in determining a NSCLC status of a subject. Such a determination can comprise detecting a level of the biomarker(s) in a sample derived from the subject. Determining the level of the biomarker in a sample may include measuring, detecting, or assaying the level of the biomarker in the sample using any suitable method, for example, the methods set forth herein. Determining the level of the biomarker in a sample may also include examining the results of an assay that measured, detected, or assayed the level of the biomarker in the sample. The method may also involve comparing the level of the biomarker in a sample with a suitable control. The method may comprise associating a latent representation of one or more polyamino acid descriptors derived from a sample associated with the subject.

Biological classification of the sample from the subject may be assisted by a classification algorithm (e.g., trained algorithm), which computes whether or not a statistically significant difference exists between the pattern of biomolecule descriptors (or representations or encodings thereof) in the sample and that from samples not associated with the biological classification. In some cases, the classification algorithm can distinguish regions of a latent space encoding biomolecule descriptors. For example, the classification algorithm can generate a first region of latent space with a first biological classification and a second region of latent space with a second biological classification. In the case of latent space representations generated with variational autoencoders, the latent space may generally be continuous, allowing for partial or intermediate classification. For example, a sample comprising biomolecule descriptors which are encoded into a region of latent space between a first region and a second region may be classified as belonging to both the first region and the second region. Alternatively, the sample may be classified as belonging to neither category.

In some embodiments, data that are generated using samples such as “known samples” can then be used to “train” a classification model. A “known sample” is a sample that has been pre-classified, e.g., classified as being derived from a normal subject, or from a subject having a certain biological classification (e.g., cancer or sub. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification method that attempts to segregate bodies of data into classes based on objective parameters present in the data. Classification methods may be either supervised or unsupervised. In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks and multilayer perceptrons (MLP), discriminant analyses (e.g., Bayesian classifier or Fischer analysis), logistic classifiers, and support vector classifiers (support vector machines (SVM)).

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Systems and methods as described herein may use more than one classification model to determine an output (e.g., cancer status of a subject). Systems and methods may comprise 1, 2, 3, 4, 5, 6, 7, 8, 9, 10, 11, 12, 13, 14, 15, 16, 17, 18, 19, 20, or more classification models. A classification model of the plurality of classification models may be trained on a particular type of data (e.g., a pattern of biomolecule descriptors observed in a sample from a subject or other health data). Alternatively, a classification model may be trained on more than one type of data. The inputs of one classification model may comprise the outputs of one or more other classification models. Additionally, a classification model may receive as its input the output of one or more classification models.

The output of classification models as described herein may comprise one or more output values which comprises one of a fixed number of possible values (e.g., a linear classifier, a logistic regression classifier, etc.) indicating a classification of the biological sample and/or the subject by the classifier. The classification model may comprise a binary classifier, such that each of the one or more output values comprises one of two values (e.g., {0, 1}, {positive, negative}, or {high-risk, low-risk}) indicating a classification of the biological sample and/or subject by the classifier. The classification model may be another type of classifier, such that each of the one or more output values comprises one of more than two values (e.g., {0, 1, 2}, {positive, negative, or indeterminate}, or {high-risk, intermediate-risk, or low-risk}) indicating a classification of the biological sample and/or subject by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's cancer state or status, and may comprise, for example, a therapeutic intervention, a duration of the therapeutic intervention, and/or a dosage of the therapeutic intervention suitable to treat a subject classified in a particular cancer-related category.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the cancer-related category of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a cancer status (e.g., type, subtype, or stage of cancer, such as stages 1, 2, or 3 of NSCLC). For example, a binary classification of samples may assign an output value of “negative” or 0 if the sample indicates that the subject has less than a 50% probability of having a cancer status. In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject belongs to a cancer status (e.g., cancer diagnosis or prognosis) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a cancer status (e.g., positive for state 1, 2, or 3 of NSCLC) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a cancer status (e.g., positive for stage 1, 2, or 3 of NSCLC) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a cancer status (e.g., positive for stage 1, 2, or 3 of NSCLC) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

In some cases, methods and systems of the present disclosure may comprise classification on latent descriptors (e.g., latent representations of biomolecule descriptors). Compared to classification of samples performed directly on biomolecule descriptors, classification of samples on latent descriptors methods and systems classifying or associating biological states based on the latent representations described herein may exhibit one or more superior properties. Classifiers operating on latent representations of biomolecule descriptors may exhibit one or more of superior performance, accuracy, PPV, NPV, clinical sensitivity, clinical specificity, or AUC relative to a classified operating directly on the input biomolecule descriptor. Without wishing to be bound by a particular theory, the embedding operation (e.g., the operation of generating the latent descriptors as described herein) may retain or amplify those parts of the input data most relevant to making high-quality classifications while discarding or diminishing those parts of the input data that are not relevant. This discrimination between important and unimportant parts of the input data may be effected by the dimensionality reduction of the encoder which is trained to amplify those parts of the data that are most informative (e.g., most relevant to reconstructing the data) while attenuating others. Accordingly, compared to methods and systems trained on biomolecule descriptors directly (e.g., rather than embeddings as described herein), methods and systems as described herein which are trained and/or classify based on embeddings of biomolecule descriptors (e.g., latent descriptors) may display superior performance. The superior performance may be measured by one or more of accuracy, positive predictive value (PPV), negative predictive value (NPV), sensitivity, specificity, area under the receiver operating characteristic curve (AUC).

The trained algorithm may be configured to classify or associate the biological category at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy classify or associate the biological category by the trained algorithm may be calculated as the percentage of independent test samples (e.g., subjects known to belong to the biological category or subjects with negative clinical test results for biological category) that are correctly identified or classified as belonging to or not belonging to the biological category. In some cases, the trained algorithm is trained on biomolecule embeddings of the present disclosure and displays an enhanced accuracy relative to a trained algorithm trained on biomolecule descriptors directly. In some cases, the accuracy may be enhanced by at least about 1%, 2%, 3%, 4%, 5%, 6%, 7%, 8%, 9%, 10%, 15%, 20%, 25%, 30%, 40%, 45%, 50%, 55%, 60%, 65%, 70%, 75%, 80%, 85%, 90%, 95%, or 99%, or more relative to the directly trained algorithm. In some cases, the accuracy may be enhanced by at most about 99%, 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1%, or less relative to the directly trained algorithm.

The trained algorithm may be configured to identify the biological category with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the biological category using the trained algorithm may be calculated as the percentage of biological samples identified or classified as having the biological category that correspond to subjects that truly belong to the biological category. In some cases, the trained algorithm is trained on biomolecule embeddings of the present disclosure and displays an enhanced PPV relative to a trained algorithm trained on biomolecule descriptors directly. In some cases, the PPV may be enhanced by at least about 1%, 2%, 3%, 4%, 5%, 6%, 7%, 8%, 9%, 10%, 15%, 20%, 25%, 30%, 40%, 45%, 50%, 55%, 60%, 65%, 70%, 75%, 80%, 85%, 90%, 95%, or 99%, or more relative to the directly trained algorithm. In some cases, the PPV may be enhanced by at most about 99%, 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1%, or less relative to the directly trained algorithm.

The trained algorithm may be configured to identify the biological category with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the biological category using the trained algorithm may be calculated as the percentage of subject datasets identified or classified as not belonging to the biological category that correspond to subjects that truly do not belong to the biological category. In some cases, the trained algorithm is trained on biomolecule embeddings of the present disclosure and displays an enhanced NPV relative to a trained algorithm trained on biomolecule descriptors directly. In some cases, the NPV may be enhanced by at least about 1%, 2%, 3%, 4%, 5%, 6%, 7%, 8%, 9%, 10%, 15%, 20%, 25%, 30%, 40%, 45%, 50%, 55%, 60%, 65%, 70%, 75%, 80%, 85%, 90%, 95%, or 99%, or more relative to the directly trained algorithm. In some cases, the NPV may be enhanced by at most about 99%, 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1%, or less relative to the directly trained algorithm.

The trained algorithm may be configured to identify the biological category with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the biological category using the trained algorithm may be calculated as the percentage of independent test samples associated with the biological category (e.g., subjects known to belong to the biological category) that are correctly identified or classified as having the biological category. In some cases, the trained algorithm is trained on biomolecule embeddings of the present disclosure and displays an enhanced sensitivity relative to a trained algorithm trained on biomolecule descriptors directly. In some cases, the sensitivity may be enhanced by at least about 1%, 2%, 3%, 4%, 5%, 6%, 7%, 8%, 9%, 10%, 15%, 20%, 25%, 30%, 40%, 45%, 50%, 55%, 60%, 65%, 70%, 75%, 80%, 85%, 90%, 95%, or 99%, or more relative to the directly trained algorithm. In some cases, the sensitivity may be enhanced by at most about 99%, 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1%, or less relative to the directly trained algorithm.

The trained algorithm may be configured to identify the biological category with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the biological category using the trained algorithm may be calculated as the percentage of independent test samples associated with absence of the biological category (e.g., subjects with negative clinical test results for the biological category) that are correctly identified or classified as not belonging to the biological category. In some cases, the trained algorithm is trained on biomolecule embeddings of the present disclosure and displays an enhanced specificity relative to a trained algorithm trained on biomolecule descriptors directly. In some cases, the specificity may be enhanced by at least about 1%, 2%, 3%, 4%, 5%, 6%, 7%, 8%, 9%, 10%, 15%, 20%, 25%, 30%, 40%, 45%, 50%, 55%, 60%, 65%, 70%, 75%, 80%, 85%, 90%, 95%, or 99%, or more relative to the directly trained algorithm. In some cases, the specificity may be enhanced by at most about 99%, 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1%, or less relative to the directly trained algorithm.

The trained algorithm may be configured to identify the biological category with an area under the receiver operating characteristic curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying datasets derived from a subject as belonging to or not belonging to the biological category. In some cases, the trained algorithm is trained on biomolecule embeddings of the present disclosure and displays an enhanced AUC relative to a trained algorithm trained on biomolecule descriptors directly. In some cases, the AUC may be enhanced by at least about 0.01, 0.02, 0.03, 0.04, 0.05, 0.06, 0.07, 0.08, 0.09, 0.10, 0.15, 0.20, 0.25, 0.30, 0.40, 0.45, 0.50, 0.55, 0.60, 0.65, 0.70, 0.75, 0.80, 0.85, 0.90, 0.95, or 0.99, or more relative to the directly trained algorithm. In some cases, the AUC may be enhanced by at most about 0.99, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.09, 0.08, 0.07, 0.06, 0.05, 0.04, 0.03, 0.02, or 0.01, or less relative to the directly trained algorithm.

The trained algorithm may be adjusted or tuned to improve one or more of the performance, accuracy, PPV, NPV, clinical sensitivity, clinical specificity, or AUC of identifying the biological category. The trained algorithm may be adjusted or tuned by adjusting parameters of the trained algorithm (e.g., a set of cutoff values used to classify a biological sample as described elsewhere herein, or weights of a neural network). The trained algorithm may be adjusted or tuned continuously during the training process or after the training process has completed.

### Biological Samples

A biological sample can comprise a single sample or a plurality of samples from a species, an individual organism, or a part of an individual organism. In some cases, the biological sample can be obtained from an individual organism. In some cases, the biological sample can comprise a plurality of samples obtained from a population of organisms. In some cases, the biological sample can comprise a gene. In some cases, the biological sample can comprise a tissue. In some cases, the biological sample can comprise an organ. In some cases, the biological sample can be obtained by performing a biopsy. In some cases, the biological sample can be obtained by performing a tissue biopsy. In some cases, the biological sample can comprise a tumor biopsy. In some cases, the biological sample can comprise a liquid biopsy. In some cases, the biological sample may be processed (e.g., lysed, blended, centrifuged, fractionated, etc.). In some cases, the biological sample may comprise media comprising biomolecules secreted by one or more cells. In some cases, the biological sample may be cell-free or substantially cell-free. In some cases, the biological sample may comprise a plurality of biomolecules. In some cases, a plurality of biomolecules may comprise lipids. In some cases, a plurality of biomolecules may comprise metabolites. In some cases, a plurality of biomolecules may comprise proteins. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof. A biological sample may comprise a member of any class of biomolecules, where “classes” may refer to any named category that defines a group of biomolecules having a common characteristic or function (e.g., proteins, nucleic acids, carbohydrates, lipids, metabolites).

In some cases, the biological sample disclosed herein comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some cases, the biological sample comprises blood, serum, or plasma, or any portion or fraction thereof. In some cases, the biological sample comprises blood. In some cases, the biological sample comprises blood or any portion or fraction thereof. In some cases, the blood is diluted. In some cases, the biological sample comprises serum. In some cases, the biological sample comprises serum or any portion or fraction thereof. In some cases, the serum is diluted. In some cases, the biological sample comprises plasma. In some cases, the biological sample comprises plasma or any portion or fraction thereof. In some cases, the plasma is diluted.

In some cases, the biological sample can comprise a cell. In some cases, a cell can refer to a basic unit of life comprising at least a cellular membrane and genetic material. In some cases, a biological sample can comprise a cell of a single-celled organism. In some cases, a biological sample can comprise a cell of a multicellular organism. In some cases, a biological sample can comprise a bacterial cell. In some cases, a biological sample can comprise a fungal cell. In some cases, a biological sample can comprise a virus-infected cell. In some cases, a biological sample can comprise a mammalian cell. In some cases, a biological sample can comprise a human cell. In some cases, a biological sample can comprise a specialized cell in a multicellular organism. In some cases, a biological sample can comprise a stem cell. In some cases, a biological sample can comprise a healthy cell. In some cases, a biological sample can comprise a cancerous cell. In some cases, a biological sample can comprise a malignant cell. In some cases, a biological sample may comprise lipids and various forms thereof. In some cases, a biological sample may comprise metabolites and various forms thereof. In some cases, a biological sample may comprise nucleic acids and various forms thereof. In some cases, a biological sample may comprise proteins and various forms thereof. In some cases, a cell is part of a plurality of cells. In some cases, the plurality of cells are cells of a same type. In some cases, the cell is of a tissue sample, an organoid, an immortalized cell line, or any combination thereof. In some cases, the cell is a stem cell. In some cases, the cell is afflicted with an infection or a mutation. In some cases, the cell is a viable cell comprising a cancer cell, an epithelial cell, a bone cell, a muscle cell, a fat cell, a tissue cell, or nerve cell. In some cases, the cancer cell is a biopsied cell of a patient. In some cases, the cell is a eukaryote or a prokaryote. In some cases, the biological sample can comprise a yeast. In some cases. In some cases, the plurality of cells is comprised in a tissue, an organoid, an organism, or a plurality of organisms. In some cases, the cell is derived from an immortalized cell line. In some cases, the cell is a HeLa cell. In some cases, the cell is a stem cell. In some cases, the cell is comprised in a primary cell culture. In some cases, the cell comprises a genetically modified cell.

A subject can comprise any living organism. In some cases, a subject can be a cell. In some cases, a subject can comprise a bacterium, a mammalian cell, a human cell, a fungal cell, a colony of bacteria, a tissue of a mammal, an organ of a mammal, a mammal, a tissue of a human, an organ of a human, a fungus, or any combination thereof. In some cases, a subject can comprise a cancer cell, a healthy cell, or both.

### Machine Learning

A machine learning model can comprise one or more of various machine learning models. In some embodiments, the machine learning model can comprise one machine learning model. In some embodiments, the machine learning model can comprise a plurality of machine learning models. In some embodiments, the machine learning model can comprise a neural network model. In some embodiments, the machine learning model can comprise a random forest model. In some embodiments, the machine learning model can comprise a manifold learning model. In some embodiments, the machine learning model can comprise a hyperparameter learning model. In some embodiments, the machine learning model can comprise an active learning model.

A graph, graph model, and graphical model can refer to a method of conceptualizing or organizing information into a graphical representation comprising nodes and edges. In some embodiments, a graph can refer to the principle of conceptualizing or organizing data, wherein the data may be stored in a various and alternative forms such as linked lists, dictionaries, spreadsheets, arrays, in permanent storage, in transient storage, and so on, and is not limited to specific embodiments disclosed herein. In some embodiments, the machine learning model can comprise a graph model.

The machine learning model can comprise a variety of manifold learning algorithms. In some embodiments, the machine learning model can comprise a manifold learning algorithm. In some embodiments, the manifold learning algorithm is principal component analysis. In some embodiments, the manifold learning algorithm is a uniform manifold approximation algorithm. In some embodiments, the manifold learning algorithm is an isomap algorithm. In some embodiments, the manifold learning algorithm is a locally linear embedding algorithm. In some embodiments, the manifold learning algorithm is a modified locally linear embedding algorithm. In some embodiments, the manifold learning algorithm is a Hessian eigenmapping algorithm. In some embodiments, the manifold learning algorithm is a spectral embedding algorithm. In some embodiments, the manifold learning algorithm is a local tangent space alignment algorithm. In some embodiments, the manifold learning algorithm is a multi-dimensional scaling algorithm. In some embodiments, the manifold learning algorithm is a t-distributed stochastic neighbor embedding algorithm (t-SNE). In some embodiments, the manifold learning algorithm is a Barnes-Hut t-SNE algorithm.

The terms reducing, dimensionality reduction, projection, component analysis, feature space reduction, latent space engineering, feature space engineering, representation engineering, or latent space embedding can refer to a method of transforming a given input data with an initial number of dimensions to another form of data that has fewer dimensions than the initial number of dimensions. In some embodiments, the terms can refer to the principle of reducing a set of input dimensions to a smaller set of output dimensions.

The term normalizing can refer to a collection of methods for adjusting a dataset to align the dataset to a common scale. In some embodiments, a normalizing method can comprise multiplying a portion or the entirety of a dataset by a factor. In some embodiments, a normalizing method can comprise adding or subtracting a constant from a portion or the entirety of a dataset. In some embodiments, a normalizing method can comprise adjusting a portion or the entirety of a dataset to a known statistical distribution. In some embodiments, a normalizing method can comprise adjusting a portion or the entirety of a dataset to a normal distribution. In some embodiments, a normalizing method can comprise adjusting the dataset so that the signal strength of a portion or the entirety of a dataset is about the same.

Converting can comprise one or more steps of various of conversions of data. In some embodiments, converting can comprise normalizing data. In some embodiments, converting can comprise performing a mathematical operation that computes a score based on a distance between 2 points in the data. In some embodiments, the distance can comprise a distance between two edges in a graph. In some embodiments, the distance can comprise a distance between two nodes in a graph. In some embodiments, the distance can comprise a distance between a node and an edge in a graph. In some embodiments, the distance can comprise a Euclidean distance. In some embodiments, the distance can comprise a non-Euclidean distance. In some embodiments, the distance can be computed in a frequency space. In some embodiments, the distance can be computed in Fourier space. In some embodiments, the distance can be computed in Laplacian space. In some embodiments, the distance can be computed in spectral space. In some embodiments, the mathematical operation can be a monotonic function based on the distance. In some embodiments, the mathematical operation can be a non-monotonic function based on the distance. In some embodiments, the mathematical operation can be an exponential decay function. In some embodiments, the mathematical operation can be a learned function.

In some embodiments, converting can comprise transforming a data in one representation to another representation. In some embodiments, converting can comprise transforming data into another form of data with less dimensions. In some embodiments, converting can comprise linearizing one or more curved paths in the data. In some embodiments, converting can be performed on data comprising data in Euclidean space. In some embodiments, converting can be performed on data comprising data in graph space. In some embodiments, converting can be performed on data in a discrete space. In some embodiments, converting can be performed on data comprising data in frequency space. In some embodiments, converting can transform data in discrete space to continuous space, continuous space to discrete space, graph space to continuous space, continuous space to graph space, graph space to discrete space, discrete space to graph space, or any combination thereof. In some embodiments, converting can comprise transforming data in discrete space into a frequency domain. In some embodiments, converting can comprise transforming data in continuous space into a frequency domain. In some embodiments, converting can comprise transforming data in graph space into a frequency domain.

In some embodiments, the methods of the disclosure further comprise reducing polyamino acid descriptors to a reduced descriptor space using a machine learning model. In some embodiments, the method further comprises clustering the reduced descriptor space to determine one or more groups of polyamino acid descriptors with similar features.

In some embodiments, reducing can comprise transforming a given input data with any initial number of dimensions to another form of data that has any number of dimensions fewer than the initial number of dimensions. In some embodiments, reducing can comprise transforming input data into another form of data with fewer dimensions. In some embodiments, reducing can comprise linearizing one or more curved paths in the input data to the output data. In some embodiments, reducing can be performed on data comprising data in Euclidean space. In some embodiments, reducing can be performed on data comprising data in graph space. In some embodiments, reducing can be performed on data in a discrete space. In some embodiments, reducing can transform data in discrete space to continuous space, continuous space to discrete space, graph space to continuous space, continuous space to graph space, graph space to discrete space, discrete space to graph space, or any combination thereof.

The terms clustering, cluster analysis, or generating modules can refer to a method of grouping samples in a dataset by some measure of similarity. Samples can be grouped in a set space, for example, element ‘a’ is in set ‘A’. Samples can be grouped in a continuous space, for example, element ‘a’ is a point in Euclidean space with distance ‘l’ away from the centroid of elements comprising cluster ‘A’. Samples can be grouped in a graph space, for example, element ‘a’ is highly connected to elements comprising cluster ‘A’. These terms can refer to the principle of organizing a plurality of elements into groups in some mathematical space based on some measure of similarity.

Clustering can comprise grouping any number of samples in a dataset by any quantitative measure of similarity. In some embodiments, clustering can comprise K-means clustering. In some embodiments, clustering can comprise hierarchical clustering. In some embodiments, clustering can comprise using random forest models. In some embodiments, clustering can comprise boosted tree models. In some embodiments, clustering can comprise using support vector machines. In some embodiments, clustering can comprise calculating one or more N−1 dimensional surfaces in N-dimensional space that partitions a dataset into clusters. In some embodiments, clustering can comprise distribution-based clustering. In some embodiments, clustering can comprise fitting a plurality of prior distributions over the data distributed in N-dimensional space. In some embodiments, clustering can comprise using density-based clustering. In some embodiments, clustering can comprise using fuzzy clustering. In some embodiments, clustering can comprise computing probability values of a data point belonging to a cluster. In some embodiments, clustering can comprise using constraints. In some embodiments, clustering can comprise using supervised learning. In some embodiments, clustering can comprise using unsupervised learning.

In some embodiments, clustering can comprise grouping samples based on similarity. In some embodiments, clustering can comprise grouping samples based on quantitative similarity. In some embodiments, clustering can comprise grouping samples based on one or more features of each sample. In some embodiments, clustering can comprise grouping samples based on one or more labels of each sample. In some embodiments, clustering can comprise grouping samples based on Euclidean coordinates. In some embodiments, clustering can comprise grouping samples based the features of the nodes and edges of each sample.

In some embodiments, comparing can comprise comparing between a first group and different second group. In some embodiments, a first or a second group can each independently be a cluster. In some embodiments, a first or a second group can each independently be a group of clusters. In some embodiments, comparing can comprise comparing between one cluster with a group of clusters. In some embodiments, comparing can comprise comparing between a first group of clusters with second group of clusters different than the first group. In some embodiments, one group can be one sample. In some embodiments, one group can be a group of samples. In some embodiments, comparing can comprise comparing between one sample versus a group of samples. In some embodiments, comparing can comprise comparing between a group of samples versus a group of samples.

### Neural Network

In some embodiments, systems and methods of the present disclosure may comprise or comprise using a neural network. The neural network may comprise various architectures, loss functions, optimization algorithms, assumptions, and various other neural network design choices. In some embodiments, the neural network comprises an encoder. In some embodiments, the neural network comprises a decoder. In some embodiments, the neural network comprises a bottleneck architecture comprising the encoder and the decoder. In some embodiments, the bottleneck architecture comprises an autoencoder. In some embodiments, the neural network comprises a language model. In some embodiments, the neural network comprises a transformer model.

Various types of layers may be used a neural network. In some embodiments, the neural network comprises a convolutional layer. In some embodiments, the neural network comprises a densely connected layer. In some embodiments, the neural network comprises a skip connection. In some embodiments, the neural network may comprise graph convolutional layers. In some embodiments, the neural network may comprise message passing layers. In some embodiments, the neural network may comprise attention layers. In some embodiments, the neural network may comprise recurrent layers. In some embodiments, the neural network may comprise a gated recurrent unit. In some embodiments, the neural network may comprise reversible layers. In some embodiments, the neural network may comprise a neural network with a bottleneck layer. In some embodiments, the neural network may comprise residual blocks. In some embodiments, the neural network may comprise one or more dropout layers. In some embodiments, the neural network may comprise one or more locally connected layers. In some embodiments, the neural network may comprise one or more batch normalization layers. In some embodiments, the neural network may comprise one or more pooling layers. In some embodiments, the neural network may comprise one or more upsampling layers. In some embodiments, the neural network may comprise one or more max-pooling layers.

In some embodiments, the neural network comprises a graph model. In some embodiments, a graph, graph model, and graphical model can refer to a method that models data in a graphical representation comprising nodes and edges. In some embodiments, the data may be stored in a various and alternative forms such as linked lists, dictionaries, spreadsheets, arrays, in permanent storage, in transient storage, and so on, and is not limited to specific embodiments disclosed herein.

In some embodiments, the neural network may comprise an autoencoder. In some embodiments, the neural network may comprise a variational autoencoder. In some embodiments, the neural network may comprise a generative adversarial network. In some embodiments, the neural network may comprise a flow model. In some embodiments, the neural network may comprise an autoregressive model.

The neural network may comprise various activation functions. In some embodiments, an activation function may be a non-linearity. In some embodiments, the neural network may comprise one or more activation functions. In some embodiments, the neural network may comprise a ReLU, softmax, tanh, sigmoid, softplus, softsign, selu, elu, exponential, LeakyReLU, or any combination thereof. Various activation functions may be used with a neural network, without departing from the inventive concepts disclosed herein.

### Training

Various loss functions can be used to train the neural network. In some embodiments, the neural network may comprise a regression loss function. In some embodiments, the neural network may comprise a logistic loss function. In some embodiments, the neural network may comprise a variational loss. In some embodiments, the neural network may comprise a prior. In some embodiments, the neural network may comprise a Gaussian prior. In some embodiments, the neural network may comprise a non-Gaussian prior. In some embodiments, the neural network may comprise a Laplacian prior. In some embodiments, the neural network may comprise a zero-inflated prior. In some case, the neural network may comprise a zero-inflated Poisson prior. In some embodiments, the neural network may comprise a zero-inflated negative binomial prior. In some embodiments, the neural network may comprise a Gaussian posterior. In some embodiments, the neural network may comprise a non-Gaussian posterior. In some embodiments, the neural network may comprise a Laplacian posterior. In some embodiments, the neural network may comprise an adversarial loss. In some embodiments, the neural network may comprise a reconstruction loss. In some embodiments, the loss functions may be formulated to optimize a regression loss, an evidence-based lower bound, a maximum likelihood, Kullback-Leibler divergence, applied with various distribution functions such as Gaussians, non-Gaussian, mixtures of Gaussians, mixtures of logistic functions, and so on.

Various optimizers can be used to train the neural network. In some embodiments, the neural network may be trained with the Adam optimizer. In some embodiments, the neural network may be trained with the stochastic gradient descent optimizer. In some embodiments, the neural network may be trained with an active learning algorithm. A neural network may be trained with various loss functions whose derivatives may be computed to update one or more parameters of the neural network. A neural network may be trained with hyperparameter searching algorithms. In some embodiments, the neural network hyperparameters are optimized with Gaussian Processes.

Various training protocols can be used while training the neural network. In some embodiments, the neural network may be trained with train/validation/test data splits. In some embodiments, the neural network may be trained with k-fold data splits, with any positive integer for k.

Training the neural network can involve providing inputs to the untrained neural network to generate predicted outputs, comparing the predicted outputs to the expected outputs, and updating the neural network's parameters to account for the difference between the predicted outputs and the expected outputs. Based on the calculated difference, a gradient with respect to each parameter may be calculated by backpropagation to update the parameters of the neural network so that the output value(s) that the neural network computes are consistent with the examples included in the training set. This process may be iterated for a certain number of iterations or until some stopping criterion is met.

The trained algorithm may be trained with a plurality of independent training samples. Each of the independent training samples may comprise a biomolecule descriptor. In the case of a variational autoencoder (VAE), the training samples may comprise individual observed biomolecule descriptors (e.g., poylamino acid descriptors, such as feature intensities) and corresponding reconstructed biomolecule descriptors. The trained algorithm may be trained, at least in part, to optimize the accuracy of the reconstruction when compared to the original input data. Such. Alternatively, or additionally,

After training the VAE, the encoder may be used to generate encodings (e.g., latent representations or latent descriptors) of biomolecule descriptors. Compared to the original or reconstructed descriptors, the latent descriptors may comprise certain properties. In some cases, the latent descriptors may comprise a reduced noise compared to the original descriptor. Without wishing to be bound by a particular theory, because the latent representation generally comprises fewer dimensions than the input feature, the autoencoder may “learn” during training to only capture in the latent representation those patterns in the input data which are significant (e.g., important for accurate reconstruction) while ignoring those that are less important. The latent space may additionally learn a continuous representation of the input data. For example, original biomolecule descriptors which are similar to one another may be close to one another in the latent space while those which are dissimilar to one another may be far apart in the latent space.

### Biomolecule Descriptors

Systems and methods as disclosed herein may ingest, operate on, transform, encode, decode, or output one or more biomolecule descriptors. Biomolecule descriptors may comprise any numerical or categorical data associated with a biomolecule. In some cases, a biomolecule descriptor comprises proteomic information as described herein. In some cases, a biomolecule descriptor comprises genomic information as described herein. In some cases, a biomolecule descriptor comprises transcriptomic information as described herein.

As used herein, “proteomic analysis”, “protein analysis”, and the like, may refer to any system or method for analyzing proteins in a sample, including the systems and methods disclosed herein. The present disclosure systems and methods for assaying using one or more surface. In some cases, a surface may comprise a surface of a high surface-area material, such as nanoparticles, particles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a plasma sample). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches, and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least 5, 6, 7, 8, 9, 10, 15, or 20 magnitudes. In some cases, a panel including any number of distinct particle types disclosed herein, may enrich and identify biomolecules over a dynamic range of at most 5, 6, 7, 8, 9, 10, 15, or 20 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N43-(Dimethylamino)propyllmethacrylamide-co42-(methacryloyloxy)ethylldimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may comprise a protein, polynucleotide, or polysaccharide, or may be comparable in size to any of the aforementioned classes of species. In some cases, a surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique biomolecules (e.g., proteins) in a biological sample (e.g., a biofluid). In some cases, a surface disclosed herein may be incubated with a biological sample to adsorb at least 1000, 2000, 3000, 4000, 5000, 6000, 7000, 8000, 9000, or 10000 unique biomolecules. In some cases, a surface disclosed herein may be incubated with a biological sample to adsorb at most 1000, 2000, 3000, 4000, 5000, 6000, 7000, 8000, 9000, or 10000 unique biomolecules. In some cases, a surface disclosed herein may be incubated with a biological sample to adsorb at least 1000, 2000, 3000, 4000, 5000, 6000, 7000, 8000, 9000, or 10000 unique biomolecule groups. In some cases, a surface disclosed herein may be incubated with a biological sample to adsorb at most 1000, 2000, 3000, 4000, 5000, 6000, 7000, 8000, 9000, or 10000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

Biomolecules collected on particles may be subjected to further analysis. In some cases, a method may comprise collecting a biomolecule adsorption layer (e.g., corona) or a subset of biomolecules from a biomolecule adsorption layer. In some cases, the collected biomolecule adsorption layer or the collected subset of biomolecules from the biomolecule adsorption layer may be subjected to further particle-based analysis (e.g., particle adsorption). In some cases, the collected biomolecule adsorption layer or the collected subset of biomolecules from the biomolecule adsorption layer may be purified or fractionated (e.g., by a chromatographic method). In some cases, the collected biomolecule adsorption layer or the collected subset of biomolecules from the biomolecule adsorption layer may be analyzed (e.g., by mass spectrometry). Analysis of the biomolecule adsorption layer (e.g., by a chromatographic method and/or mass spectrometry) may generate biomolecule descriptors indicative of the composition of the biomolecule adsorption layer for use in the methods and systems (e.g., for generating embeddings or classifying samples) described herein.

In some cases, the panels disclosed herein can be used to identify a number of proteins, peptides, protein groups, or protein classes using a protein analysis workflow described herein (e.g., a protein corona analysis workflow). In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, a biomolecule descriptor comprises a peptide (e.g., polyamino acid). In some cases, a peptide may be a tryptic peptide. In some cases, a biomolecule descriptor comprises a tryptic peptide. In some cases, a peptide may be a semi-tryptic peptide. In some cases, a biomolecule descriptor comprises a semi-tryptic peptide. In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct biomolecule adsorption layers corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence). In some cases, a biomolecule descriptor comprises a feature intensity. In some cases, a biomolecule descriptor comprises a protein or protein group.

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least 2 proteins, 5 proteins, 10 proteins, 20 proteins, 40 proteins, 60 proteins, 80 proteins, 100 proteins, 150 proteins, 200 proteins, or more. In some cases, a biomolecule descriptor comprises a protein class.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, and other protein separation techniques. In some cases, a biomolecule descriptor comprises proteomic data.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids.

In some cases, an assay may rapidly generate and analyze proteomic data. In some cases, beginning with an input biological sample (e.g., a buccal or nasal smear, plasma, or tissue), a method of the present disclosure may generate and analyze proteomic data in less than about 1, 2, 3, 4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg g, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

In some cases, proteomic information or data can refer to information about substances comprising a peptide and/or a protein component. In some cases, proteomic information may comprise primary structure information, secondary structure information, tertiary structure information, or quaternary information about the peptide or a protein. In some cases, proteomic information may comprise information about protein-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, a biomolecule descriptor comprises proteomic information

In some cases, proteomic information may comprise information about a single cell, a tissue, an organ, a system of tissues and/or organs (such as cardiovascular, respiratory, digestive, or nervous systems), or an entire multicellular organism. In some cases, proteomic information may comprise information about an individual (e.g., an individual human being or an individual bacterium), or a population of individuals (e.g., human beings with diagnosed with cancer or a colony of bacteria). Proteomic information may comprise information from various forms of life, including forms of life from the Archaea, the Bacteria, the Eukarya, the Protozoa, the Chromista, the Plantae, the Fungi, or from the Animalia. In some cases, proteomic information may comprise information from viruses.

In some cases, proteomic information may comprise information relating exons and introns in the code of life. In some cases, proteomic information may comprise information regarding variations in the primary structure, variations in the secondary structure, variations in the tertiary structure, or variations in the quaternary structure of peptides and/or proteins. In some cases, proteomic information may comprise information regarding variations in the expression of exons, including alternative splicing variations, structural variations, or both. In some cases, proteomic information may comprise conformation information, post-translational modification information, chemical modification information (e.g., phosphorylation), cofactor (e.g., salts or other regulatory chemicals) association information, or substrate association information of peptides and/or proteins.

In some cases, proteomic information may comprise information related to various proteoforms in a sample. In some cases, a proteomic information may comprise information related to peptide variants, protein variants, or both. In some cases, a proteomic information may comprise information related to splicing variants, allelic variants, post-translation modification variants, or any combination thereof. In some cases, a biomolecule descriptor comprises proteoform data.

In some cases, splicing variant (in some cases also referred to as “alternative splicing” variants, “differential splicing” variants, or “alternative RNA splicing” variants) may refer to a protein that is expressed by an alternative splicing process. In some cases, an alternative splicing process may express one or more splicing variants from a set of exons via different combinations of exons. In some cases, a combination may comprise a different sequence of exons compared to another combination. In some cases, a combination may comprise a different subset of exons compared to another combination. In some cases, a splicing variant may comprise a reordered amino acid sequence of another splicing variant.

In some cases, an allelic variant may refer to a protein that is expressed from a gene comprising a mutation compared to a reference gene. In some cases, the reference gene may be the gene of a cell, an individual, or a population of individuals. In some cases, the mutation may be a base substitution, a base deletion, or a base insertion of a genetic sequence of the gene compared to a genetic reference of the reference gene. In some cases, an allelic variant may comprise an amino acid substitution in an amino acid sequence of another allelic variant.

In some cases, a post-translation modification may refer to a protein that is modified after expression. A protein may be modified by various enzymes. In some cases, an enzyme that can modify a protein may be a kinase, a protease, a ligase, a phosphatase, a transferase, a phosphotransferase, or any other enzyme for performing the any one of modifications disclosed herein.

In some cases, peptide variants or protein variants may comprise a post-translation modification. In some cases, the post-translational modification comprises acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

In some cases, proteomic information may be encoded as digital information. In some cases, the proteomic information may comprise one or more elements that represents the proteomic information. In some cases, an element may represent a primary structure information, secondary structure information, tertiary structure information, or quaternary information about a peptide or a protein. In some cases, an element may represent protein-ligand interactions for a peptide or a protein. In some cases, an element may represent a source of a peptide or protein (e.g., a specific cell, tissue, organ, organism, individual, or population of individuals). In some cases, an element may represent a type of proteoform. In some cases, an element may be a number, a vector, an array, or any other datatypes provided herein. In some cases, a biomolecule descriptor comprises the element or a plurality of elements.

As used herein, “genomic analysis”, “nucleic acid analysis”, and the like, may refer to any system or method for analyzing proteins in a sample, including the systems and methods disclosed herein. The present disclosure describes various compositions and methods for analyzing (e.g., detecting or sequencing) nucleic acids. In some cases, genotypic (or genomic) information may be obtained using some of the compositions and methods of the present disclosure. In some cases, genotypic information can refer to information about substances comprising a nucleotide and/or a nucleic acid component. In some cases, genotypic information may comprise epigenetic information. In some cases, epigenetic information may comprise histone modification, DNA methylation, accessibility of different regions in a genome, dynamics changes thereof, or any combination thereof. In some cases, genotypic information may comprise primary structure information, secondary structure information, tertiary structure information, or quaternary information about a nucleic acid. In some cases, genotypic information may comprise information about nucleic acid-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, genotypic information may comprise a rate or prevalence of apoptosis of a healthy cell or a diseased cell. In some cases, genotypic information may comprise a state of a cell, such as a healthy state or a diseased state. In some cases, genotypic information may comprise chemical modification information of a nucleic acid molecule. In some cases, a chemical modification may comprise methylation, demethylation, amination, deamination, acetylation, oxidation, oxygenation, reduction, or any combination thereof. In some cases, genotypic information may comprise information regarding from which type of cell a biological sample originates. In some cases, genotypic information may comprise information about an untranslated region of nucleic acids.

In some cases, genotypic information may comprise information about a single cell, a tissue, an organ, a system of tissues and/or organs (such as cardiovascular, respiratory, digestive, or nervous systems), or an entire multicellular organism. In some cases, genotypic information may comprise information about an individual (e.g., an individual human being or an individual bacterium), or a population of individuals (e.g., human beings with diagnosed with cancer or a colony of bacteria). Genotypic information may comprise information from various forms of life, including forms of life from the Archaea, the Bacteria, the Eukarya, the Protozoa, the Chromista, the Plantae, the Fungi, or from the Animalia. In some cases, genotypic information may comprise information from viruses.

In some cases, genotypic information may comprise information relating to exons and introns in the code of life. In some cases, genotypic information may comprise information regarding variations or mutations in the primary structure of nucleic acids, including base substitutions, deletions, insertions, or any combination thereof. In some cases, genotypic information may comprise information regarding the inclusion of non-canonical nucleobases in nucleic acids. In some cases, genotypic information may comprise information regarding variations or mutations in epigenetics.

In some cases, genotypic information may comprise information regarding variations in the primary structure, variations in the secondary structure, variations in the tertiary structure, or variations in the quaternary structure of peptides and/or proteins that one or more nucleic acids encode.

In some cases, the set of nucleic acids comprise an exome of the biological sample. In some cases, the set of nucleic acids comprise a genome, an epigenome, a transcriptome, a metabolome, a secretome, or any combination thereof. In some cases, the set of nucleic acids comprises a portion of the exome of the biological sample. In some cases, the set of nucleic acids comprise a portion of the genome, an epigenome, a transcriptome, a metabolome, a secretome, or any combination thereof. In some cases, the genotypic information comprises an exome sequence of the biological sample. In some cases, the genotypic information comprises one or more sequences of the genome, an epigenome, a transcriptome, a metabolome, a secretome, or any combination thereof.

Various sequencing methods and various sequencing reagents may be used to obtain genotypic information. In some cases, the sequencing methods disclosed herein may comprise enriching one or more nucleic acid molecules from a sample. This may comprise enrichment in solution, enrichment on a sensor element (e.g., a particle), enrichment on a substrate (e.g., a surface of an Eppendorf tube), or selective removal of a nucleic acid (e.g., by sequence-specific affinity precipitation). Enrichment may comprise amplification, including differential amplification of two or more different target nucleic acids. Differential amplification may be based on sequence, CG-content, or post-transcriptional modifications, such as methylation state. In some cases, enrichment may comprise hybridization methods, such as pull-down methods. For example, a substrate partition may comprise immobilized nucleic acids capable of hybridizing to nucleic acids of a particular sequence, and thereby capable of isolating particular nucleic acids from a complex biological solution. In some cases, hybridization may target genes, exons, introns, regulatory regions, splice sites, reassembly genes, among other nucleic acid targets. In some cases, hybridization can utilize a pool of nucleic acid probes that are designed to target multiple distinct sequences, or to tile a single sequence.

Enrichment may comprise a hybridization reaction and may generate a subset of nucleic acid molecules from a biological sample. Hybridization may be performed in solution, on a substrate surface (e.g., a wall of a well in a microwell plate), on a sensor element, or any combination thereof. A hybridization method may be sensitive for single nucleotide polymorphisms. For example, a hybridization method may comprise molecular inversion probes.

Enrichment may also comprise amplification. Suitable amplification methods include polymerase chain reaction (PCR), solid-phase PCR, RT-PCR, qPCR, multiplex PCR, touchdown PCR, nanoPCR, nested PCR, hot start PCR, helicase-dependent amplification, loop mediated isothermal amplification (LAMP), self-sustained sequence replication, nucleic acid sequence-based amplification, strand displacement amplification, rolling circle amplification, ligase chain reaction, and any other suitable amplification technique.

The sequencing may target a specific sequence or region of a genome. The sequencing may target a type of sequence, such as exons. In some cases, the sequencing comprises exome sequencing. In some cases, the sequencing comprises whole exome sequencing. The sequencing may target chromatinated or non-chromatinated nucleic acids. The sequencing may be sequence-nonspecific (e.g., provide a reading regardless of the target sequence). The sequencing may target a polymerase accessible region of the genome. The sequencing may target nucleic acids localized in a part of a cell, such as the mitochondria or the cytoplasm. The sequencing may target nucleic acids localized in a cell, tissue, or an organ. The sequencing may target RNA, DNA, any other nucleic acid, or any combination thereof.

‘Nucleic acid’ may refer to a polymeric form of nucleotides of any length, in single-, double- or multi-stranded form. A nucleic acid may comprise any combination of ribonucleotides, deoxyribonucleotides, and natural and non-natural analogues thereof, including 5-bromouracil, peptide nucleic acids, locked nucleotides, glycol nucleotides, threose nucleotides, dideoxynucleotides, 3′-deoxyribonucleotides, dideoxyribonucleotides, 7-deaza-GTP, fluorophores-bound nucleotides, thiol containing nucleotides, biotin linked nucleotides, methyl-7-guanosine, methylated nucleotides, inosine, thiouridine, pseudourdine, dihydrouridine, queuosine, and wyosine. A nucleic acid may comprise a gene, a portion of a gene, an exon, an intron, messenger RNA (mRNA), transfer RNA (tRNA), ribosomal RNA (rRNA), short interfering RNA (siRNA), short-hairpin RNA (shRNA), micro-RNA (miRNA), a ribozyme, cDNA, a recombinant nucleic acid, a branched nucleic acid, a plasmid, cell-free DNA (cfDNA), cell-free RNA (cfRNA), genomic DNA, mitochondrial DNA (mtDNA), circulating tumor DNA (ctDNA), long non-coding RNA, telomerase RNA, Piwi-interacting RNA, small nuclear RNA (snRNA), small interfering RNA, YRNA, circular RNA, small nucleolar RNA, or pseudogene RNA. A nucleic acid may comprise a DNA or RNA molecule. A nucleic acid may also have a defined 3-dimensional structure. In some cases, a nucleic acid may comprise a non-canonical nucleobase or a nucleotide, such as hypoxanthine, xanthine, 7-methylguanine, 5,6-dihydrouracil, 5-methylcytosine, or any combination thereof. Nucleic acids may also comprise non-nucleic acid molecules.

A nucleic acid may be derived from various sources. In some cases, a nucleic acid may be derived from an exosome, an apoptotic body, a tumor cell, a healthy cell, a virtosome, an extracellular membrane vesicle, a neutrophil extracellular trap (NET), or any combination thereof.

A nucleic acid may comprise various lengths. In some cases, a nucleic acid may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 nucleotides. In some cases, a nucleic acid may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 nucleotides.

Various reagents may be used for sequencing. In some cases, a reagent may comprise primers, oligonucleotides, switch oligonucleotides, adapters, amplification adapters, polymerases, dNTPs, co-factors, buffers, enzymes, ionic co-factors, ligase, reverse transcriptase, restriction enzymes, endonucleases, transposase, protease, proteinase K, DNase, RNase, lysis agents, lysozymes, achromopeptidase, lysostaphin, labiase, kitalase, lyticase, inhibitors, inactivating agents, chelating agents, EDTA, crowding agents, reducing agents, DTT, surfactants, TritonX-IOO, Tween 20, sodium dodecyl sulfate, sarcosyl, or any combination thereof.

Various methods for sequencing nucleic acids may be used. In some cases, sequencing may comprise sequencing a whole genome or portions thereof. Sequencing may comprise sequencing a whole genome, a whole exome, portions thereof (e.g., a panel of genes, including potentially coding and non-coding regions thereof). Sequencing may comprise sequencing a transcriptome or portion thereof. Sequencing may comprise sequencing an exome or portion thereof. Sequencing coverage may be optimized based on analytical or experimental setup, or desired sequencing footprint. In some cases, a nucleic acid sequencing method may comprise high-throughput sequencing, next-generation sequencing, flow sequencing, massively-parallel sequencing, shotgun sequencing, single-molecule real-time sequencing, ion semiconductor sequencing, electrophoretic sequencing, pyrosequencing, sequencing by synthesis, combinatorial probe anchor synthesis sequencing, sequencing by ligation, nanopore sequencing, GenapSys sequencing, chain termination sequencing, polony sequencing, 454 pyrosequencing, reversible terminated chemistry sequencing, heliscope single molecule sequencing, tunneling currents DNA sequencing, sequencing by hybridization, clonal single molecule array sequencing, sequencing with MS, DNA-seq, RNA-seq, ATAC-seq, methyl-seq, ChIP-seq, or any combination thereof. The sequencing methods of the present disclosure may involve sequence analysis of RNA. RNA sequences or expression levels may be analyzed by using a reverse transcription reaction to generate complementary DNA (cDNA) molecules from RNA for sequencing or by using reverse transcription polymerase chain reaction for quantification of expression levels. The sequencing methods of the present disclosure may detect RNA structural variants and isoforms, such as splicing variants and structural variants. The sequencing methods of the present disclosure may quantify RNA sequences or structural variants. In some cases, a sequencing may method comprise spatial sequencing, single-cell sequencing or any combination thereof.

In some cases, nucleic acids may be processed by standard molecular biology techniques for downstream applications. In some cases, nucleic acids may be prepared from nucleic acids isolated from a sample of the present disclosure. In some cases, the nucleic acids may subsequently be attached to an adaptor polynucleotide sequence, which may comprise a double stranded nucleic acid. In some cases, the nucleic acids may be end repaired prior to attaching to the adaptor polynucleotide sequences. In some cases, adaptor polynucleotides may be attached to one or both ends of the nucleotide sequences. In some cases, the same or different adaptor may be bound to each end of the fragment, thereby producing an “adaptor-nucleic acid-adaptor” construct. In some cases, a plurality of the same or different adaptor may be bound to each end of the fragment. In some cases, different adaptors may be attached to each end of the nucleic acid when adaptors are attached to both ends of the nucleic acid.

In some cases, an oligonucleotide tag complementary to a sequencing primer may be incorporated with adaptors attached to a target nucleic acid. For analysis of multiple samples, different oligonucleotide tags complementary to separate sequencing primers may be incorporated with adaptors attached to a target nucleic acid.

In some cases, an oligonucleotide index tag may also be incorporated with adaptors attached to a target nucleic acid. In cases in which deletion products are generated from a plurality of polynucleotides prior to hybridizing the deletion products to a nucleic acid immobilized on a structure (e.g., a sensor element such as a particle), polynucleotides corresponding to different nucleic acids of interest may first be attached to different oligonucleotide tags such that subsequently generated deletion products corresponding to different nucleic acids of interest may be grouped or differentiated. Consequently, deletion products derived from the same nucleic acid of interest may have the same oligonucleotide index tag such that the index tag identifies sequencing reads derived from the same nucleic acid of interest. Likewise, deletion products derived from different nucleic acids of interest may have different oligonucleotide index tags to allow them to be grouped or differentiated such as on a sensor element. Oligonucleotide index tags may range in length from about 5, 10, 15, 20, 25, 30, 35, 40, 45, 50, 75, to 100 nucleotides or base pairs, or any length in between.

In some cases, the oligonucleotide index tags may be added separately or in conjunction with a primer, primer binding site or other component. Conversely, a pair-end read may be performed, wherein the read from the first end may comprise a portion of the sequence of interest and the read from the other (second) end may be utilized as a tag to identify the fragment from which the first read originated.

In some cases, a sequencing read may be initiated from the point of incorporation of the modified nucleotide into an extended capture probe. In some cases, a sequencing primer may be hybridized to extended capture probes or their complements, which may be optionally amplified prior to initiating a sequence read and extended in the presence of natural nucleotides. In some cases, extension of the sequencing primer may stall at the point of incorporation of the first modified nucleotide incorporated in the template, and a complementary modified nucleotide may be incorporated at the point of stall using a polymerase capable of incorporating a modified nucleotide (e.g. TiTaq polymerase). In some cases, a sequencing read may be initiated at the first base after the stall or point of modified nucleotide incorporation. In a sequencing-by-synthesis method, a sequencing read may be initiated at the first base after the stall or point of modified nucleotide incorporation.

The present disclosure describes methods and compositions related to nucleic acid (polynucleotide) sequencing. Some methods of the present disclosure may provide for identification and quantification of nucleic acids in a subject or a sample. In some cases, the nucleotide sequence of a portion of a target nucleic acid or fragment thereof may be determined using a variety of methods and devices. Examples of sequencing methods include electrophoretic, sequencing by synthesis, sequencing by ligation, sequencing by hybridization, single-molecule sequencing, and real time sequencing methods. In some cases, the process to determine the nucleotide sequence of a target nucleic acid or fragment thereof may be an automated process. In some amplification reactions, capture probes may function as primers permitting the priming of a nucleotide synthesis reaction using a polynucleotide from the nucleic acid sample as a template. In this way, information regarding the sequence of the polynucleotides supplied to the array may be obtained. In some cases, polynucleotides hybridized to capture probes on the array may serve as sequencing templates if primers that hybridize to the polynucleotides bound to the capture probes and sequencing reagents are further supplied to the array.

Nucleic acid analysis methods may generate paired end reads on nucleic acid clusters. In some cases, a nucleic acid cluster may be immobilized on a sensor element, such as a surface. In some cases, paired end sequencing facilitates reading both the forward and reverse template strands of each cluster during one paired-end read. In some cases, template clusters may be amplified on the surface of a substrate (e.g. a flow-cell) by bridge amplification and sequenced by paired primers sequentially. Upon amplification of the template strands, a bridged double stranded structure may be produced. This may be treated to release a portion of one of the strands of each duplex from the surface. The single stranded nucleic acid may be available for sequencing, primer hybridization and cycles of primer extension. After the first sequencing run, the ends of the first single stranded template may be hybridized to the immobilized primers remaining from the initial cluster amplification procedure. The immobilized primers may be extended using the hybridized first single strand as a template to resynthesize the original double stranded structure. The double stranded structure may be treated to remove at least a portion of the first template strand to leave the resynthesized strand immobilized in single stranded form. The resynthesized strand may be sequenced to determine a second read, whose location originates from the opposite end of the original template fragment obtained from the fragmentation process.

Nucleic acid sequencing may be single-molecule sequencing or sequencing by synthesis. Sequencing may be massively parallel array sequencing (e.g., Illumina™ sequencing), which may be performed using template nucleic acid molecules immobilized on a support, such as a flow cell. A high-throughput sequencing method may sequence simultaneously (or substantially simultaneously) at least about 10,000, 100,000, 1 million, 10 million, 100 million, 1 billion, or more polynucleotide molecules. Sequencing methods may include, but are not limited to: pyrosequencing, sequencing-by synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PacBio, SOLiD, Ion Torrent, or Nanopore platforms. Sequencing may comprise a first-generation sequencing method, such as Maxam-Gilbert or Sanger sequencing, or a high-throughput sequencing (e.g., next-generation sequencing or NGS) method.

The sequencing methods of the present disclosure may be able to detect germline susceptibility loci, somatic single nucleotide polymorphisms (SNPs), small insertion and deletion (indel) mutations, copy number variations (CNVs) and structural variants (SVs).

Furthermore, the sequencing methods of the present disclosure may quantify a nucleic acid, thus allowing sequence variations within an individual sample may be identified and quantified (e.g., a first percent of a gene is unmutated and a second percent of a gene present in a sample contains an indel).

Nucleic acid analysis methods may comprise physical analysis of nucleic acids collected from a biological sample. A method may distinguish nucleic acids based on their mass, post-transcriptional modification state (e.g., capping), histonylation, circularization (e.g., to detect extrachromosomal circular DNA elements), or melting temperature. For example, an assay may comprise restriction fragment length polymorphism (RFLP) or electrophoretic analysis on DNA collected from a biological sample. In some cases, post-transcriptional modification may comprise 5′ capping, 3′ cleavage, 3′ polyadenylation, splicing, or any combination thereof.

Nucleic acid analysis may also include sequence-specific interrogation. An assay for sequence-specific interrogation may target a particular sequence to determine its presence, absence or relative abundance in a biological sample. For example, an assay may comprise a southern blot, qPCR, fluorescence in situ hybridization (FISH), array-Comparative Genomic Hybridization (array-CGH), quantitative fluorescence PCR (QF-PCR), nanopore sequencing, sequencing by hybridization, sequencing by synthesis, sequencing by ligation, or capture by nucleic acid binding moieties (e.g., single stranded nucleotides or nucleic acid binding proteins) to determine the presence of a gene of interest (e.g., an oncogene) in a sample collected from a subject. An assay may also couple sequence specific collection with sequencing analysis. For example, an assay may comprise generating a particular sticky-end motif in nucleic acids comprising a specific target sequence, ligating an adaptor to nucleic acids with the particular sticky-end motif, and sequencing the adaptor-ligated nucleic acids to determine the presence or prevalence of mutations in a gene of interest.

The present disclosure provides various systems and methods for analyzing (e.g., detecting or sequencing) nucleic acids. In some cases, genotypic (or genomic) information may be obtained using some of the compositions and methods of the present disclosure. In some cases, genotypic information can refer to information about substances comprising a nucleotide and/or a nucleic acid component. In some cases, genotypic information may comprise epigenetic information. In some cases, epigenetic information may comprise histone modification, DNA methylation, accessibility of different regions in a genome, dynamics changes thereof, or any combination thereof. In some cases, genotypic information may comprise primary structure information, secondary structure information, tertiary structure information, or quaternary information about a nucleic acid. In some cases, genotypic information may comprise information about nucleic acid-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, genotypic information may comprise a rate or prevalence of apoptosis of a healthy cell or a diseased cell. In some cases, genotypic information may comprise a state of a cell, such as a healthy state or a diseased state. In some cases, genotypic information may comprise chemical modification information of a nucleic acid molecule. In some cases, a chemical modification may comprise methylation, demethylation, amination, deamination, acetylation, oxidation, oxygenation, reduction, or any combination thereof. In some cases, genotypic information may comprise information regarding from which type of cell a biological sample originates. In some cases, genotypic information may comprise information about an untranslated region of nucleic acids.

In some cases, genotypic information may comprise information about a single cell, a tissue, an organ, a system of tissues and/or organs (such as cardiovascular, respiratory, digestive, or nervous systems), or an entire multicellular organism. In some cases, genotypic information may comprise information about an individual (e.g., an individual human being or an individual bacterium), or a population of individuals (e.g., human beings with diagnosed with cancer or a colony of bacteria). Genotypic information may comprise information from various forms of life, including forms of life from the Archaea, the Bacteria, the Eukarya, the Protozoa, the Chromista, the Plantae, the Fungi, or from the Animalia. In some cases, genotypic information may comprise information from viruses. In some cases, genotypic information may comprise information relating exons and introns in the code of life. In some cases, genotypic information may comprise information regarding variations or mutations in the primary structure of nucleic acids, including base substitutions, deletions, insertions, or any combination thereof. In some cases, genotypic information may comprise information regarding the inclusion of non-canonical nucleobases in nucleic acids. In some cases, genotypic information may comprise information regarding variations or mutations in epigenetics.

In some cases, genotypic information may comprise information regarding variations in the primary structure, variations in the secondary structure, variations in the tertiary structure, or variations in the quaternary structure of peptides and/or proteins that one or more nucleic acids encode.

In some cases, a genomic variant may be detected using an assay. In some cases, a genomic variant can refer to a nucleic acid sequence originating from a DNA address(es) in a sample that comprises a sequence that is different a nucleic acid sequence originating from the same DNA address(es) in a reference sample. In some cases, a genomic variant may comprise a mutation such as an insertion mutation, deletion mutations, substitution mutation, copy number variations, transversions, translocations, inversion, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, abnormal changes in nucleic acid methylation infection, chromosal lesions, DNA lesions, or any combination thereof. In some cases, a set of genomic variants may comprise a single nucleotide polymorphism (SNP).

### Computer Systems

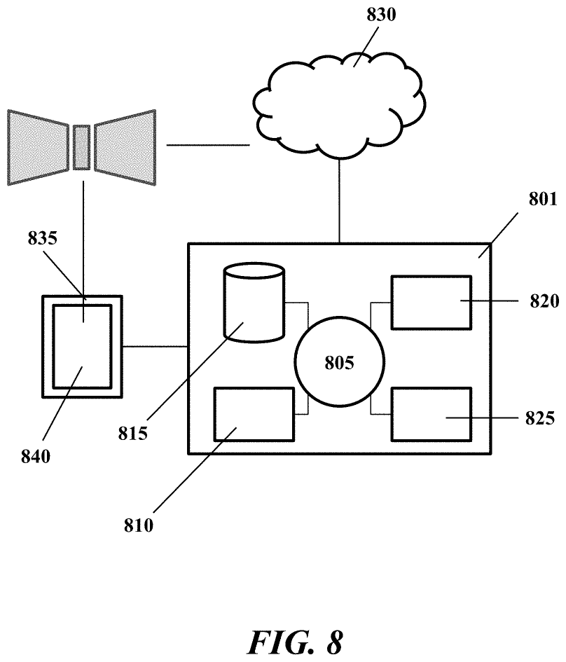

The present disclosure provides computer systems that are programmed to implement methods of the disclosure. FIG. 8 shows a computer system 801 that is programmed or otherwise configured to, for example, process proteomic data, train a neural network, or visualize embeddings.

The computer system 801 may regulate various aspects of analysis, calculation, and generation of the present disclosure, such as, for example, processing proteomic data, training a neural network, or visualizing embeddings. The computer system 801 may be an electronic device of a user or a computer system that is remotely located with respect to the electronic device. The electronic device may be a mobile electronic device.

The computer system 801 includes a central processing unit (CPU, also “processor” and “computer processor” herein) 805, which may be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 may be a data storage unit (or data repository) for storing data. The computer system 801 may be operatively coupled to a computer network (“network”) 830 with the aid of the communication interface 820. The network 830 may be the Internet, an internet and/or extranet, or an intranet and/or extranet that is in communication with the Internet.

The network 830 in some cases is a telecommunication and/or data network. The network 830 may include one or more computer servers, which may enable distributed computing, such as cloud computing. For example, one or more computer servers may enable cloud computing over the network 830 (“the cloud”) to perform various aspects of analysis, calculation, and generation of the present disclosure, such as, for example, processing proteomic data, training a neural network, or visualizing embeddings. Such cloud computing may be provided by cloud computing platforms such as, for example, Amazon Web Services (AWS), Microsoft Azure, Google Cloud Platform, and IBM cloud. The network 830, in some cases with the aid of the computer system 801, may implement a peer-to-peer network, which may enable devices coupled to the computer system 801 to behave as a client or a server.

The CPU 805 may comprise one or more computer processors and/or one or more graphics processing units (GPUs). The CPU 805 may execute a sequence of machine-readable instructions, which may be embodied in a program or software. The instructions may be stored in a memory location, such as the memory 810. The instructions may be directed to the CPU 805, which may subsequently program or otherwise configure the CPU 805 to implement methods of the present disclosure. Examples of operations performed by the CPU 805 may include fetch, decode, execute, and writeback.

The CPU 805 may be part of a circuit, such as an integrated circuit. One or more other components of the system 801 may be included in the circuit. In some cases, the circuit is an application specific integrated circuit (ASIC).

The storage unit 815 may store files, such as drivers, libraries and saved programs. The storage unit 815 may store user data, e.g., user preferences and user programs. The computer system 801 in some cases may include one or more additional data storage units that are external to the computer system 801, such as located on a remote server that is in communication with the computer system 801 through an intranet or the Internet.

The computer system 801 may communicate with one or more remote computer systems through the network 830. For instance, the computer system 801 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 801 via the network 830.

Methods as described herein may be implemented by way of machine (e.g., computer processor) executable code stored on an electronic storage location of the computer system 801, such as, for example, on the memory 810 or electronic storage unit 815. The machine executable or machine readable code may be provided in the form of software. During use, the code may be executed by the processor 805. In some cases, the code may be retrieved from the storage unit 815 and stored on the memory 810 for ready access by the processor 805. In some situations, the electronic storage unit 815 may be precluded, and machine-executable instructions are stored on memory 810.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 801, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 may include or be in communication with an electronic display 835 that comprises a user interface (UI) 840 for providing, for example, processing proteomic data, training a neural network, or visualizing embeddings. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 805. The algorithm can, for example, processing proteomic data, training a neural network, or visualizing embeddings.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Using a Variational Autoencoder to Create Biomolecule Embeddings

This example describes a large cohort proteomics data analysis that provides novel biological insights, empowering disease classification and biomarker discovery.

A variational autoencoder was used to train on proteomic data to create embeddings for (i) visualization, and (ii) classification of biological states associated with the proteomic data. The proteomic data was obtained from a cohort of samples comprising healthy subjects and subjects afflicted with a disease. The variational autoencoder (VAE) neural network was built on top of open-source python libraries.

FIG. 1 schematically illustrates an architecture diagram of the VAE neural network used. Each rectangle denotes a fully connected block. Laplace and Zero Inflated Negative Binomial distributions were used as the posterior and prior distributions, respectively, to encourage sparsity and to model sparsity.

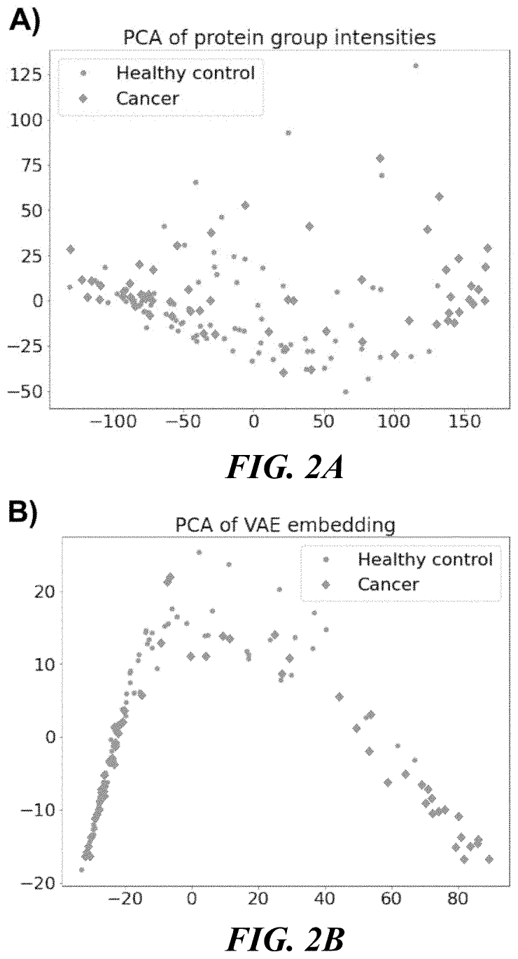

FIGS. 2A-2B illustrate PCA projection of the cohort. PCA of protein group intensity matrix (FIG. 2A) and PCA of the VAE embedding (FIG. 2B) are shown. The VAE embedding shows the ability of the VAE to separate the cohort based on disease status.

### Example 2: Analysis of Proteomic Data Using DIA-NN

Previously, a deep interrogation of plasma was conducted with samples from 141 subjects: 61 early-stage NSCLC subjects and 80 non-cancer controls. 2,499 plasma proteins were identified, with 1,992 plasma proteins that were present in ≥25% of the samples. Leveraging this data, a biomarker classifier was created for distinguishing samples from subject afflicted with NSCLC against controls with area under the receiver operating characteristic curve (AUC-ROC) of 0.91.

In this example, the data were re-analyzed with the more sensitive DIA-NN software to enhance protein depth while preserving the accuracy of the classifier. The cohort was analyzed with DIA-NN v1.8 in single group-run in library free mode against standard human Uniprot proteome using the “-relaxed-prof-inf” option. Differential expression of proteins was computed using DIA-NN estimated “log₁₀(1+intensities) and Welch's t-test. Functional enrichment analysis was performed using g:Profiler (version e104_eg51_p15_3922dba) with g:SCS multiple testing correction method applying significant threshold of 0.05. Cohort classification was performed with several machine learning classifiers applied to protein intensities as well as the learned embeddings from the previous example.

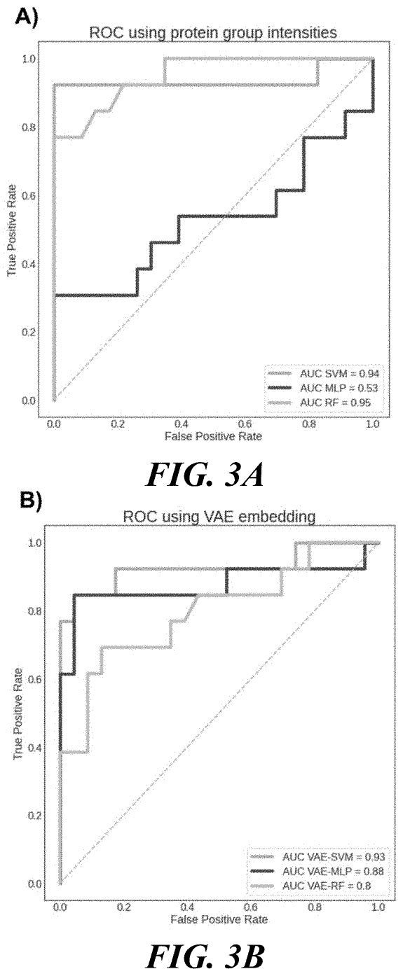

FIGS. 3A-3B show classification performance using protein intensities and VAE embedding, respectively. Classification of healthy and early-stage cancer individuals was achieved at an AUC-ROC of up to about 0.95 (FIG. 3A). Classification using VAE embedding (FIG. 3B) showed greater stability across classifiers (random forest: RF; support vector machine: SVM; multilayer perceptron: MLP) compared to using raw protein group intensities.

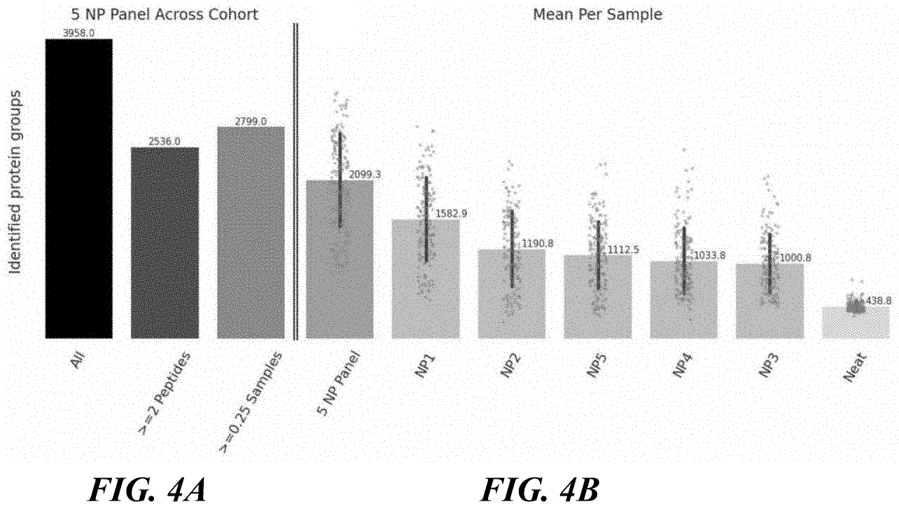

FIGS. 4A-4B show the distribution of identified protein group counts. Using DIA-NN, about 4,000 protein groups were identified across the cohort with the majority being supported by multiple peptides and being found in multiple samples.

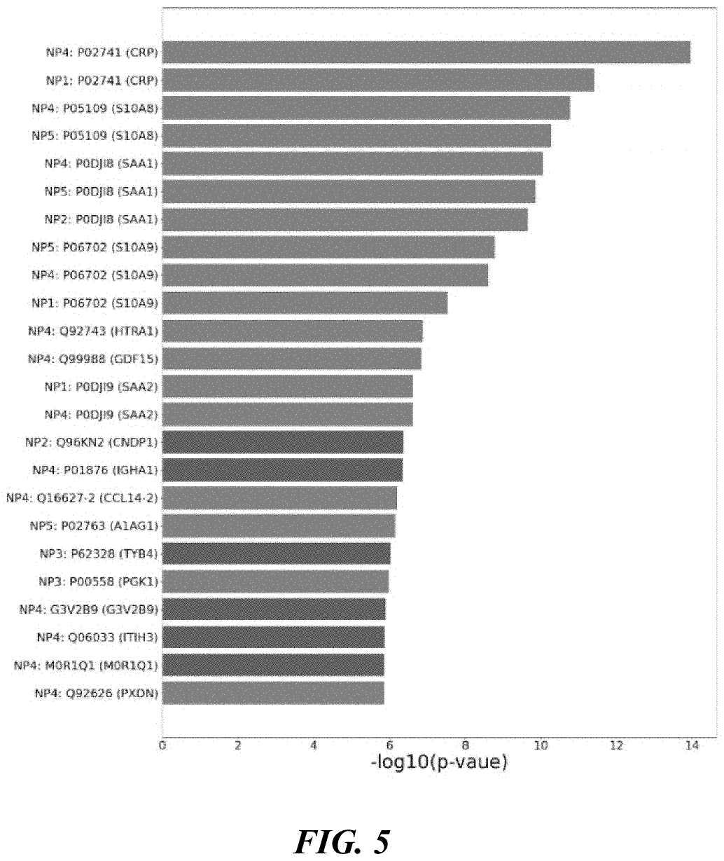

FIG. 5 shows differentially expressed protein groups across the cohort. The Welsh's t-test on protein intensities resulted in 17 unique protein groups that were significantly differentially expressed after Bonferroni correction, which may serve as putative biomarkers. The biomarkers are listed in Table 1 below.

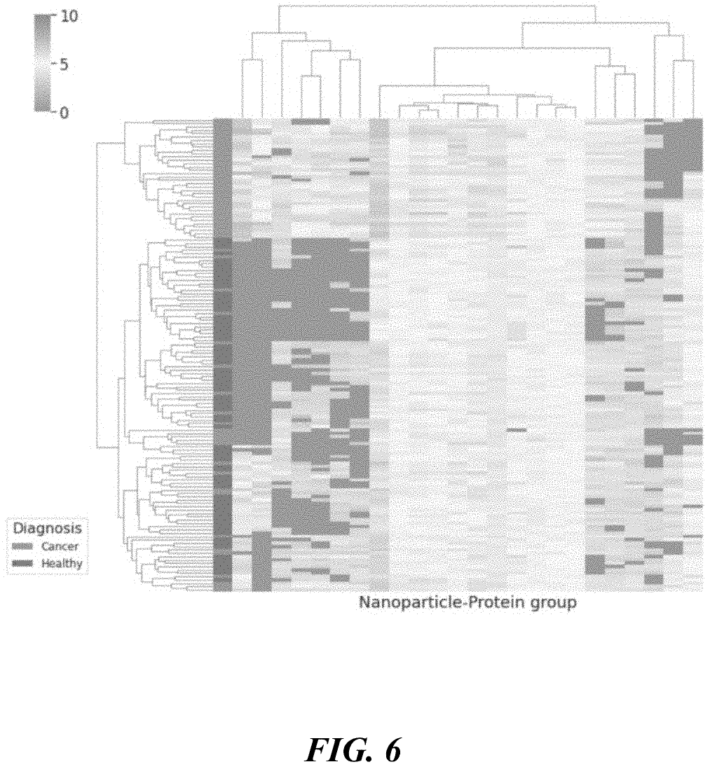

FIG. 6 shows hierarchical clustering on the differentially expressed protein intensities, which depicts a distinct separation of cancer and healthy subjects.

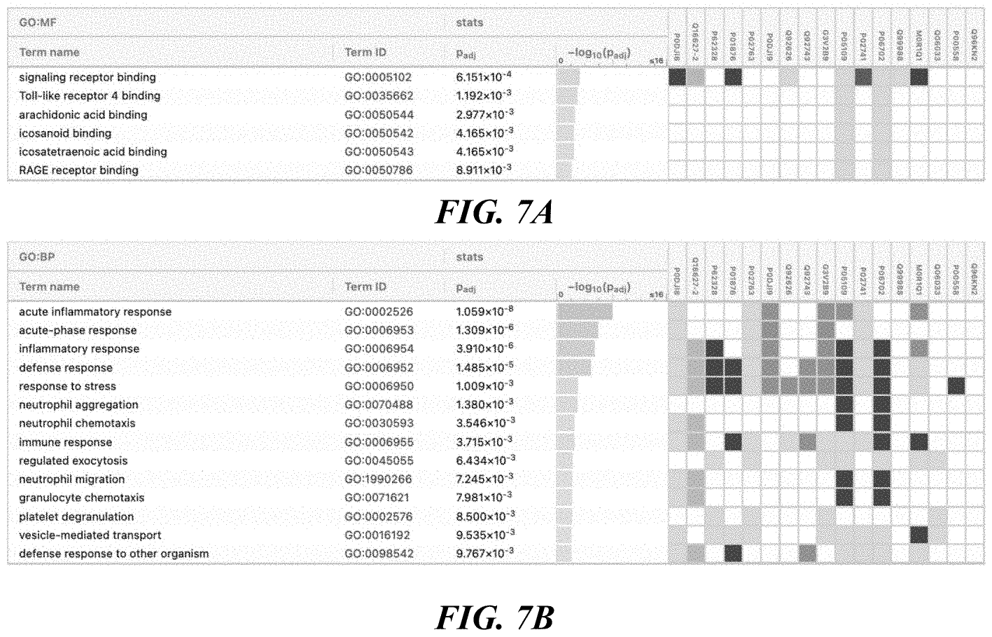

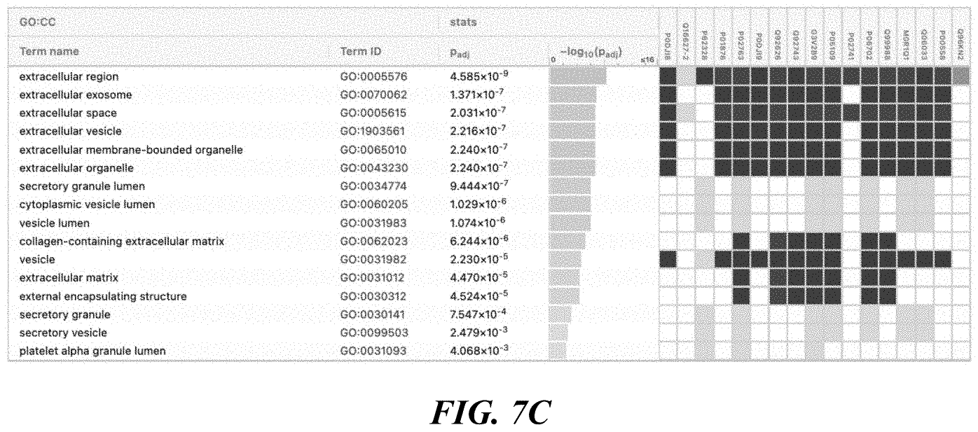

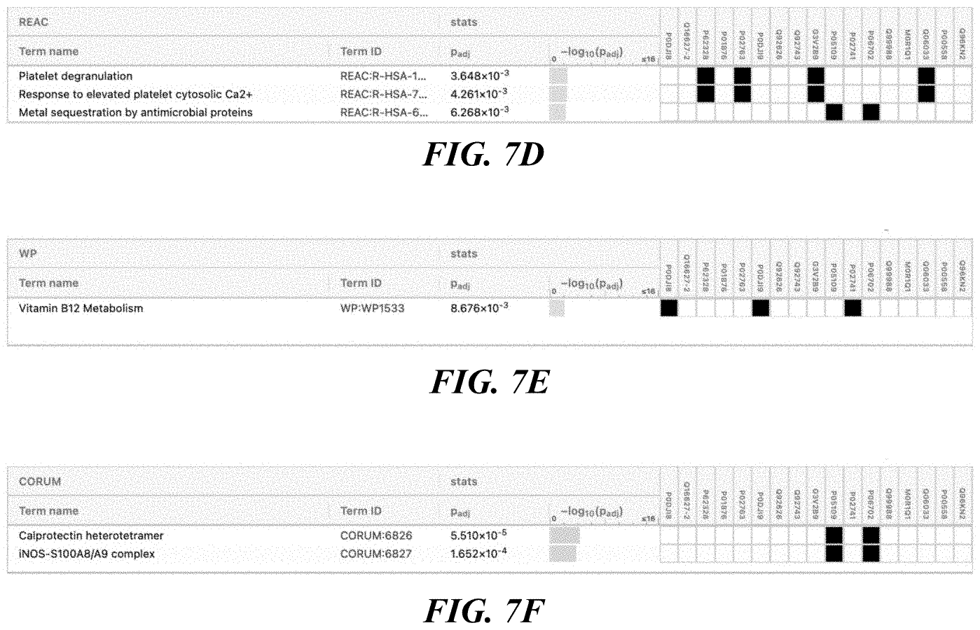

FIGS. 7A-7F depict functional enrichment of the 17 differentially expressed proteins in Table 1 across functional categories after correction for multi-correction testing using g:Profiler's g:SCS multiple hypothesis testing method that accounts for relationships between ontology terms. As illustrated in FIGS. 7A-7F, the differentially expressed proteins in Table 1 are associated with various biochemical and biological categories of clinical significance.

TABLE 1
List of biomarkers identified in previous study and current study.

| Importance Rank | Previous Study | Current Study |
|---|---|---|
| 1 | Q8NI99 (ANGPTL6) | P02741 (CRP)* |
| 2 | P58335 (ANTXR2) | P05109 (S10A8)* |
| 3 | P55774 (CCL18) | P0DJI8 (SAA1)* |
| 4 | Q92743 (HTRA1) | P06702 (S10A9)* |
| 5 | Q71U36 (TUBA1A) | Q92743 (HTRA1) |
| 6 | Q92626 (PXDN) | Q99988 (GDF15)* |
| 7 | P18827 (SDC1) | P0DJI9 (SAA2) |
| 8 | P05387 (RPLP2) | Q96KN2 (CNDP1)* |
| 9 | P0DJI9 (SAA2) | P01876 (IGHA1)* |
| 10 | P13224 (GP1BB) | Q16627-2 (CCL14-2)* |
| 11 | P15907 (ST6GAL1) | P02763 (A1AG1)* |
| 12 | Q6P988 (NOTUM) | P62328 (TYB4)* |
| 13 | O75339 (CILP) | P00558 (PGK1)* |
| 14 | P60660 (MYL6) | G3V2B9 (G3V2B9)* |
| 15 | Q13162 (PRDX4) | Q06033 (ITIH3)* |
| 16 | P01782 (IGHV3-9) | M0R1Q1 (M0R1Q1)* |
| 17 | P22692 (IGFBP4) | Q92626 (PXDN) |

Asterisk (*) denotes newly identified biomarkers.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1. A method for training a neural network, comprising:
(a) providing a neural network comprising:
i) an input layer configured to receive at least a polyamino acid descriptor;
ii) a latent layer configured to output at least a latent descriptor, wherein the latent layer is connected to the input layer, and wherein the latent descriptor comprises sufficiently fewer dimensions than the polyamino acid descriptor such that at least a portion of information in the polyamino acid descriptor is filtered in the latent descriptor;
iii) an output layer configured to output at least a reconstruction of the polyamino acid descriptor, wherein the output layer is connected to the latent layer; and
iv) at least one parameter;
(b) providing training data comprising a plurality of polyamino acid descriptors, wherein the plurality of polyamino acid descriptors comprises at least one value for a polyamino acid in association with a given assay method; and
(c) training the neural network, by (i) inputting at least the plurality of polyamino acid descriptors at the input layer of the neural network, (ii) outputting a plurality of latent descriptors at the latent layer and a plurality of reconstructions at the output layer, and (iii) optimizing at least one loss function based at least in part on the plurality of latent descriptors and the plurality of reconstructions by adjusting the at least one parameter, such that the neural network learns a latent space comprising a denoised embedding for the plurality of polyamino acid descriptors.

2. The method of claim 1, wherein the output layer outputs a plurality of parameters for a probability distribution.

3. The method of claim 2, wherein the probability distribution is a zero inflated distribution.

4. The method of claim 3, wherein the zero inflated distribution is a zero inflated negative binomial distribution.

5. (canceled)

6. The method of claim 1, wherein the polyamino acid descriptor comprises at least 100 dimensions.

7. The method of claim 1, wherein the latent descriptor comprises at most about 50% of the number of dimensions in the polyamino acid descriptor.

8. (canceled)

9. The method of claim 1, wherein at least about 10% of values in the plurality of polyamino acid descriptors in the training data are zero.

10. (canceled)

11. The method of claim 1, wherein the latent layer outputs a plurality of parameters for a posterior distribution.

12. The method of claim 11, wherein the posterior distribution is comprises a higher kurtosis than a normal distribution.

13. The method of claim 1, wherein the at least one loss function comprises a Kullbeck-Leibler divergence loss function based at least in part on a difference between a sum of posterior distributions parameterized by the plurality of parameters and a prior distribution.

14. (canceled)

15. The method of claim 13, wherein the prior distribution comprises a higher kurtosis than a normal distribution.

16. (canceled)

17. (canceled)

18. The method of claim 1, wherein the given assay method comprises contacting a plurality of biomolecules with a given surface.

19. The method of claim 18, wherein the given surface is a surface of a particle.

20. The method of claim 18, wherein the given assay method comprises (i) performing mass spectrometry on cleaved derivatives of the plurality of biomolecules to obtain a plurality of peptide spectral signals and (ii) processing the plurality of peptide spectral signals to obtain a plurality of peptide identifications, wherein the plurality of polyamino acid descriptors comprises the plurality of peptide identifications.

21. The method claim 18, wherein the given assay method comprises (i) performing mass spectrometry on cleaved derivatives of the plurality of biomolecules to obtain a plurality peptide spectral signals (ii) processing the plurality of peptide spectral signals to obtain a plurality of peptide identifications and (iii) processing the plurality of peptide identifications to obtain a plurality of intensities for plurality of protein or protein group identification, wherein the plurality of polyamino acid descriptors comprises the plurality of protein or protein group identifications.

22. The method of claim 1, further comprising classifying at least a first set of latent descriptors from a second set of latent descriptors, wherein the first set of latent descriptors is associated with a first biological state and the second set of latent descriptors is associated with a second biological state.

23. (canceled)

24. (canceled)

25. The method of claim 1, wherein the at least one polyamino acid descriptor comprises an identification of at least one protein or protein group.

26. The method of claim 1, wherein the at least one polyamino acid descriptor comprises an identification of at least one peptide.

27. (canceled)

28. (canceled)

29. (canceled)

30. A computer-implemented system comprising:
a digital processing device comprising:
at least one processor, an operating system configured to perform executable instructions, a memory, and a computer program including instructions executable by the digital processing device that, upon execution by the at least one processor, implements a method to learn a denoised embedding, the method comprising:
(a) providing, in the memory, a neural network comprising:
i) an input layer configured to receive at least a polyamino acid descriptor;
ii) a latent layer configured to output at least a latent descriptor, wherein the latent layer is connected to the input layer, and wherein the latent descriptor comprises sufficiently fewer dimensions than the polyamino acid descriptor such that at least a portion of information in the polyamino acid descriptor is filtered in the latent descriptor;
iii) an output layer configured to output at least a reconstruction of the polyamino acid descriptor, wherein the output layer is connected to the latent layer; and
iv) at least one parameter;
(b) providing, in the memory, training data comprising a plurality of polyamino acid descriptors, wherein the plurality of polyamino acid descriptors comprises at least one value for a polyamino acid in association with a given assay method; and
(c) training the neural network, by (i) inputting at least the plurality of polyamino acid descriptors at the input layer of the neural network, (ii) outputting a plurality of latent descriptors at the latent layer and a plurality of reconstructions at the output layer, and (iii) optimizing at least one loss function based at least in part on the plurality of latent descriptors and the plurality of reconstructions by adjusting the at least one parameter, such that the neural network learns a latent space comprising a denoised embedding for the plurality of polyamino acid descriptors.

31. A method for determining a biological state associated with a polyamino acid descriptor, comprising:
(a) receiving the polyamino acid descriptor comprising at least one dimension representing a polyamino acid association with a given assay method;
(b) generating, in a latent space, a latent descriptor based at least in part on the polyamino acid descriptor, and wherein the latent descriptor comprises sufficiently fewer dimensions than the polyamino acid descriptor such that at least a portion of information in the polyamino acid descriptor is lost in the latent descriptor; and
(c) determining, based at least in part on the latent descriptor, the biological state associated with the polyamino acid descriptor.

32.-65. (canceled)
